(19) United States
(12) Patent Application Publication
(10) Pub. No.: US 2025/0018019 A1
(43) Pub. Date: Jan. 16, 2025

(54) TREATMENT OF INTERFERON DRIVEN INFLAMMATION

(71) Applicant: The Provost, Fellows, Foundation Scholars and the other members of Board, of the College of the Holy, Dublin (IE)

(72) Inventors: Luke O’Neill, Dublin (IE); Christian Peace, Dublin (IE); Alexander Hooftman, Dublin (IE); Dylan Ryan, Dublin (IE)

(21) Appl. No.: 18/765,977

(22) Filed: Jul. 8, 2024

Related U.S. Application Data
(60) Provisional application No. 63/525,780, filed on Jul. 10, 2023.

(51) Int. Cl. A61K 38/51; A61K 48/00; A61P 37/06

(52) U.S. Cl. CPC A61K 38/51; A61K 48/0033; A61P 37/06

(57) ABSTRACT

Compositions and methods for regulating immunometabolism and preventing and/or treating interferon-driven inflammation, including anti-inflammatory agents which upregulate fumarate hydratase enzymatic activity and/or expression of fumarate hydratase protein, and methods for preventing and/or treating inflammation in a patient in need thereof, wherein the methods comprise: administering an effective amount of a fumarate hydratase modulating agent to the patient.

### CROSS-REFERENCE TO RELATED APPLICATIONS

This application claims the benefit of priority from U.S. Provisional Patent Application 63/525,780 filed Jul. 10, 2023, the entire disclosure of which is herein incorporated by reference in its entirety.

### INCORPORATION BY REFERENCE OF SEQUENCE LISTING FILE

This application contains a Sequence Listing which has been submitted electronically in .XML format. Said .XML file, created on Jul. 1, 2024, is named “370259.US.02.xml” and is 2,000 bytes in size. The sequence listing contained in this .XML file is part of the specification and is hereby incorporated by reference in its entirety.

### TECHNICAL FIELD

This invention relates to the field of immunometabolism and the innate immune system. The invention is directed to the prevention and/or treatment of interferon driven inflammation by upregulating the fumarate hydratase metabolic pathway and/or associated expression.

### BACKGROUND TO THE INVENTION

Metabolic pathways play a central role in regulating immune cell function and activation, with dysregulation across these pathways impacting immune response, specifically the innate immune system, and contributing to the progression of chronic autoimmune and inflammatory disease.

Accordingly, the study of immune metabolism aims to provide further insights into the function of immune cells, including how they exert immune responses. A better understanding of the metabolic mechanisms of disease progression aims to reveal new ways to target inflammatory diseases such as autoimmune diseases, chronic viral infections, and cancer.

In recent years, targeting interferons has become an attractive therapeutic target. For example, activation of TLR, such as TLR7, and RIG-1 via MDA5 leads to cytokine responses, e.g. with release of interferons and activation of specified immune cells. Known treatments include monoclonal antibodies which target the interferon receptor and prevent its signalling, and small-molecule inhibitors of interferon receptor signalling. These types of treatments have been approved in a variety of diseases including systemic lupus erythematosus (SLE) and asthma.

### SUMMARY

An object of the present invention is the provision of a new immunometabolism therapeutic target and associated treatment methodology.

Metabolic rewiring underlies macrophage effector functions¹⁻³, but the mechanisms involved remain incompletely defined. Here, using unbiased metabolomics and stable isotope-assisted tracing, we show induction of an inflammatory aspartate-argininosuccinate shunt following LPS stimulation. The shunt, supported by increased ASS1 expression, also leads to increased cytosolic fumarate levels and fumarate-mediated protein succination. Pharmacologic inhibition and genetic ablation of the TCA cycle enzyme FH further elevates intracellular fumarate levels, suppresses mitochondrial respiration, and increases mitochondrial membrane potential. RNA sequencing and proteomic analysis demonstrates profound inflammatory effects resulting from FH inhibition. Of note, acute FH inhibition suppresses IL-10 expression leading to increased TNF-α secretion, an effect recapitulated by fumarate esters. Unexpectedly, FH inhibition, but not fumarate esters, also increases IFN-β production through mechanisms that are driven by mitochondrial RNA (mtRNA) release and activation of the RNA sensors TLR7 and RIG-I/MDA5. This effect is recapitulated endogenously when FH is suppressed following prolonged LPS stimulation. Furthermore, cells from SLE patients also exhibit FH suppression, indicating a potential pathogenic role for this process in human disease. We therefore identify a protective role for FH in maintaining appropriate macrophage cytokine and interferon responses.

In one aspect, this disclosure provides a method for prevention and/or treatment of inflammation in a patient in need thereof, the method comprising
administering an effective amount of a fumarate hydratase modulating agent to said patient.

The methods include those, wherein the inflammation is interferon driven inflammation. The methods also include those, wherein said patient has low levels of fumarate hydratase expression compared to a healthy subject and/or wherein said patient has an interferon-driven disease associated with decreased FH activity and/or expression.

In preferred embodiments of the methods, the fumarate hydratase modulating agent may upregulate fumarate hydratase activity and/or expression to reduce or prevent macrophage, cytokine and/or interferon production in a patient in need thereof. In particularly preferred embodiments, the fumarate hydratase modulating agent may upregulate fumarate hydratase activity and/or expression to reduce or prevent type 1 interferon (type 1 IFN) production in a patient in need thereof. In some preferred embodiments, the type 1 interferon (type 1 IFN) may include IFN-β.

Some embodiments of the method include those, wherein the fumarate hydratase modulating agent may upregulate fumarate hydratase activity and/or expression to reduce or prevent interleukin-10 (IL-10) and/or tumor necrosis factor (TNF) production in a patient in need thereof. In at least some preferred embodiments of the method, the fumarate hydratase modulating agent may upregulate fumarate hydratase activity and/or expression to reduce or prevent cytosolic mitochondrial RNA release and/or signalling via ssRNA and dsRNA receptors, including ssRNA receptor TLR7 and/or dsRNA receptors MDA5 and RIG-I.

In embodiments of the method, some preferred fumarate hydratase modulating agent may be selected from one or more of the following: fumarate hydratase; a small molecule activator of fumarate hydratase; a pharmaceutical composition comprising fumarate hydratase or a small molecule activator of fumarate hydratase; a viral vector comprising a fumarate hydratase gene or mRNA thereof; or a non-viral vector, such as a lipid nanoparticle, comprising a fumarate hydratase gene or mRNA thereof.

In some embodiments, the method may further comprise the delivery of the fumarate hydratase modulating agent to inflammatory cells or pro-inflammatory cells of said patient. In some embodiments, the method may further comprise the delivery of the fumarate hydratase gene or mRNA thereof to inflammatory cells or pro-inflammatory cells of said patient. In some embodiments of the method, the inflammatory or pro-inflammatory cells may include macrophages.

Some embodiments of the method may include treatment of inflammation of the skin, kidneys and/or joints in said patient. Some embodiments of the method may include treatment of auto-immune diseases such as systemic lupus erythematosus (SLE), asthma, arthritis, sepsis and/or covid-19 in said patient. Some embodiments of the method may include treatment of chronic inflammation in said patient. In particularly preferred embodiments, the method may include treatment of sepsis during bacterial or viral infections in said patient.

In another aspect, this disclosure relates to an anti-inflammatory agent for use in upregulating fumarate hydratase activity and/or expression to reduce or prevent interferon production, wherein the anti-inflammatory agent may be selected from one or more of the following: fumarate hydratase; a small molecule activator of fumarate hydratase; a pharmaceutical composition comprising said small molecule activator of fumarate hydratase; a viral vector comprising a fumarate hydratase gene or mRNA thereof; or a non-viral vector, such as a lipid nanoparticle, comprising a fumarate hydratase gene or mRNA thereof.

### BRIEF DESCRIPTION OF THE DRAWINGS

The patent or patent application contains at least one drawing executed in color. Copies of this patent or patent application publication with color drawings will be provided by the Office upon request and payment of the necessary fee.

FIG. 1—LPS stimulation drives fumarate accumulation via glutamine anaplerosis and an aspartate-argininosuccinate shunt

Metabolite abundance (a,d) and bioenergetic ratios (b) in non-stimulated (NS) versus LPS-stimulated BMDMs (n=3; LPS 4 h; argininosuccinate (P=0.000044), fumarate (P=0.000141), malate (P=0.000219)). c, Respirometry as measured by oxygen consumption rate (OCR) of NS and LPS-stimulated BMDMs (n=6 (NS); n=8 (LPS); LPS 4 h). n=technical replicates from 1 experiment performed with 3 pooled biological replicates. Data are mean±s.d. e, Ass1 and Fh1 gene expression with LPS time-course (n=9; 24 h (P=0.000729), 48 h (P=0.000001)). f, Quantitative proteomics of aspartate-argininosuccinate shunt enzymes in NS and LPS-stimulated BMDMs (n=4, LPS 24 h; ASS1 (P=0.000156)). g, FH protein levels with LPS time-course (n=1). h, Fumarate levels following LPS stimulation with or without aminooxyacetic acid (AOAA) pre-treatment (1 h) (n=6, LPS 4 h). i, Schematic of metabolic changes occurring during early-phase TCA cycle rewiring. Created with BioRender.com. b,d-f,h, Data are mean±s.e.m. n=biological replicates unless stated otherwise. P values calculated using two-tailed Student's t-test for paired comparisons or one-way analysis of variance (ANOVA) for multiple comparisons.

FIG. 2—FH inhibition increases bioenergetic stress, fumarate levels and mitochondrial membrane potential

Bioenergetic ratios (a) and heatmap of top 50 differentially abundant metabolites (c) in BMDMs pre-treated with vehicle (DMSO), FH inhibitor (FHIN1) or dimethyl fumarate (DMF) (n=3; LPS 4 h; ATP/ADP (P=0.000004), phosphocreatine/creatine (P=0.00000001)). b, Respirometry of BMDMs pre-treated with DMSO, FHIN1 or DMF (n=8; LPS 4 h). n=technical replicates from 1 experiment performed with 3 pooled biological replicates. Data are mean±s.d. d, PCA plot of metabolomics in BMDMs pre-treated with DMSO, FHIN1 or DMF or (n=3; LPS 4 h). e, Fumarate levels in BMDMs pre-treated with DMSO or FHIN1 (n=9; LPS 4 h). f, Fumarate and 2SC levels in Fh1^{+/+} and Fh1^{-/-} BMDMs (n=3; 96 h EtOH/TAM; LPS 4 h). g, Mean fluorescence intensity (MFI) of CellROX staining in BMDMs pre-treated with DMSO, FHIN1 or DMF (n=3 (CellROX); n=4 (TMRM); LPS 4 h). h, MFI of TMRM staining in BMDMs pre-treated with DMSO, FHIN1 or DMF or Fh1^{+/+} and Fh1^{-/-} BMDMs (n=4 (DMSO/FHIN1/DMF); n=3 (Fh1^{+/+} and Fh1^{-/-}); 72 h EtOH/TAM; LPS 4 h). i, Aconitate/citrate ratio following LPS stimulation with or without FHIN1 or DMF pre-treatment (n=3; LPS 4 h). j, GSH and GSSG levels following LPS stimulation with or without FHIN1 or DMF pre-treatment (n=3; LPS 4 h). a, e-j Data are mean±s.e.m. n=biological replicates unless stated otherwise. P values calculated using two-tailed Student's t-test for paired comparisons or one-way or ANOVA for multiple comparisons.

FIG. 3—FH activity is required to maintain appropriate cytokine responses GSEA (a) and overrepresentation analysis (ORA) (b) of RNAseq in BMDMs pre-treated with FHIN1 or DMSO (n=3; LPS 4 h). c, IL-10 and TNF-α release from DMSO-FHIN1- or DMF-pre-treated BMDMs (n=6; LPS 4 h; FHIN1/IL-10 (P=0.0000024), DMF/IL-10 (P=0.0000018), FHIN1/TNF (P=0.000001)). d, II10 and Tnfa expression in DMSO- or MMF-pre-treated BMDMs (n=3; LPS 4 h). e, Enrichment map plot of shared significantly decreased genes in FHIN1- and DMF-pre-treated BMDMs (n=3; LPS 4 h). f, II10 expression in DMSO- FHIN1- or DMF-pre-treated BMDMs in the presence of NAC (n=3; LPS 4 h). g, c-Fos activity in DMSO- FHIN1- or DMF-pre-treated BMDMs (n=3; LPS 4 h; DMF (P=0.0000298)). h, TNF-α release from BMDMs pre-treated with anti-CD210 antibody (1 h) (n=4; LPS 4 h). Western blot for STAT3 and phospho-STAT3 (i) and TNF-α release (j) from DMSO- FHIN1- or DMF-pre-treated BMDMs and co-treated with IL-10 (n=3, LPS 4 h; DMF (P=0.000163)). k, II10expression and IL-10 release in Fh1^{+/+} and Fh1^{-/31} (n=5 or 2)/Fh1^{+/-} (n=2) BMDMs (EtOH/TAM 72 h; LPS 4 h; II10 (P=0.000055)). I, TNF-α release from Fh1^{+/-} and Fh1^{-/-} (n=5)/Fh1^{+/-} (n=2) BMDMs (EtOH/TAM 72 h; LPS 4 h). m, IL10 and TNFA expression in DMSO- or FHIN1-pre-treated human PBMCs (n=8, LPS 4 h; FHIN1 (P=0.00000008)). n, IL10 and TNFA expression in DMSO- or FHIN1- pre-treated human macrophages (n=3, LPS 4 h; FHIN1 (P=0.000028)). c,d,f-h,j-n Data are mean±s.e.m. i, 1 representative blot of 3 shown. n=biological replicates. P values calculated using two-tailed Student's t-test for paired comparisons or one-way ANOVA for multiple comparisons.

FIG. 4—FH impairment triggers IFN-β release via a mtRNA-driven retrograde response

a, Volcano plot of DMSO- or FHIN1-pre-treated BMDMs (n=3; LPS 4 h). b, IFN-β release from DMSO-, FHIN1- or DMF-pre-treated BMDMs (n=6; LPS 4 h; FHIN1 (P=0.000004)). c, Ifnb1 in DMSO- or MMF-pre-treated BMDMs (n=3; LPS 4 h). d, IFN-β release from BMDMs treated with EtBr (6 days), before pre-treatment with DMSO or FHIN1 (n=6; LPS 4 h). e, Cytosolic D-loop in DNA and RNA in DMSO- or FHIN1-pre-treated BMDMs (n=4 for mtDNA, n=5 for mtRNA; LPS 4 h). f, dsRNA in DMSO- or FHIN1-pre-treated BMDMs (n=3; LPS 4 h). Scale bar=20 μm. g, Ifnb1 with Tlr7 silencing in DMSO- or FHIN1-pre-treated BMDMs (n=3; LPS 4 h). h, IFN-β with Ddx58 or Ifih1 silencing in DMSO- or FHIN1-pre-treated BMDMs (n=7; LPS 4 h).

i, MAVS in DMSO- or FHIN1-pre-treated BMDMs (n=3; LPS 4 h). j, IFN-β in Fh1^{+/+} and Fh1^{-/-} BMDMs (n=3, EtOH/TAM 72 h; LPS 4 h). k, dsRNA in Fh1^{+/+} and Fh1^{-/-} BMDMs (n=3; EtOH/TAM 72 h; LPS 4 h). Scale bar=20 μm. I, Ifnb1 with Ddx58 or Ifih1 silencing (n=3). m, Serum IFN-β of FHIN1- or DMF-treated mice prior to PBS or LPS injection (n=5 (PBS); n=10 (FHIN1/LPS); n=11 (Vehicle/LPS); n=12 (DMF/LPS)). n, IFN-β release from DMSO-, FHIN1 -or DMF-pre-treated human PBMCs (n=3; LPS 4 h). o, FH in whole blood from healthy controls and SLE patients (n=30; P=0.0000005). b-e,g,h,j,i-o, Data are mean±s.e.m. f,i,k, 1 representative blot or image of 3 experiments shown. n=biological replicates. P values calculated using two-tailed Student's t-test for paired comparisons, one-way ANOVA for multiple comparisons.

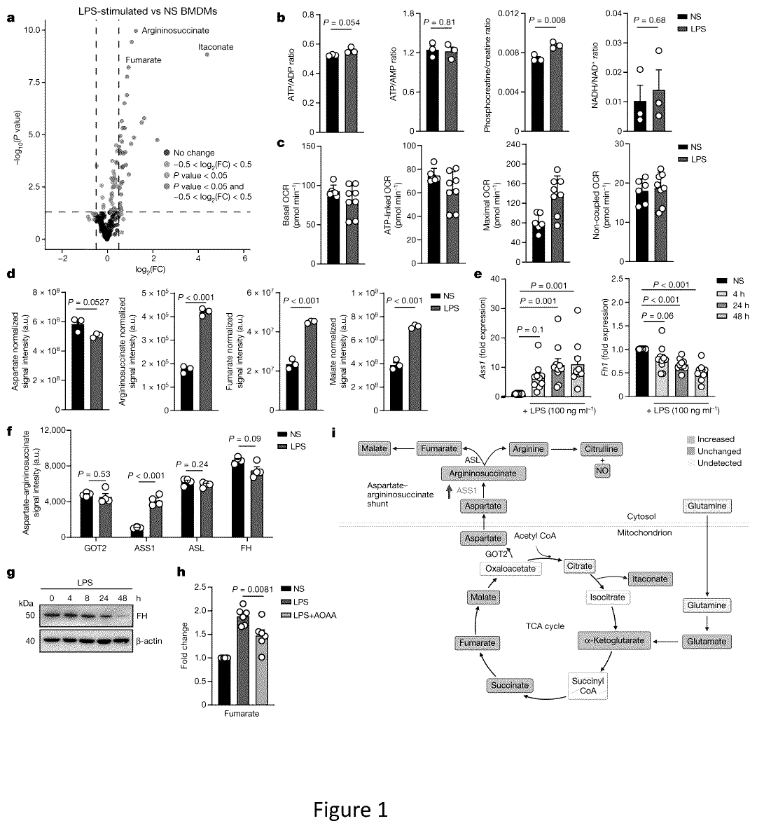

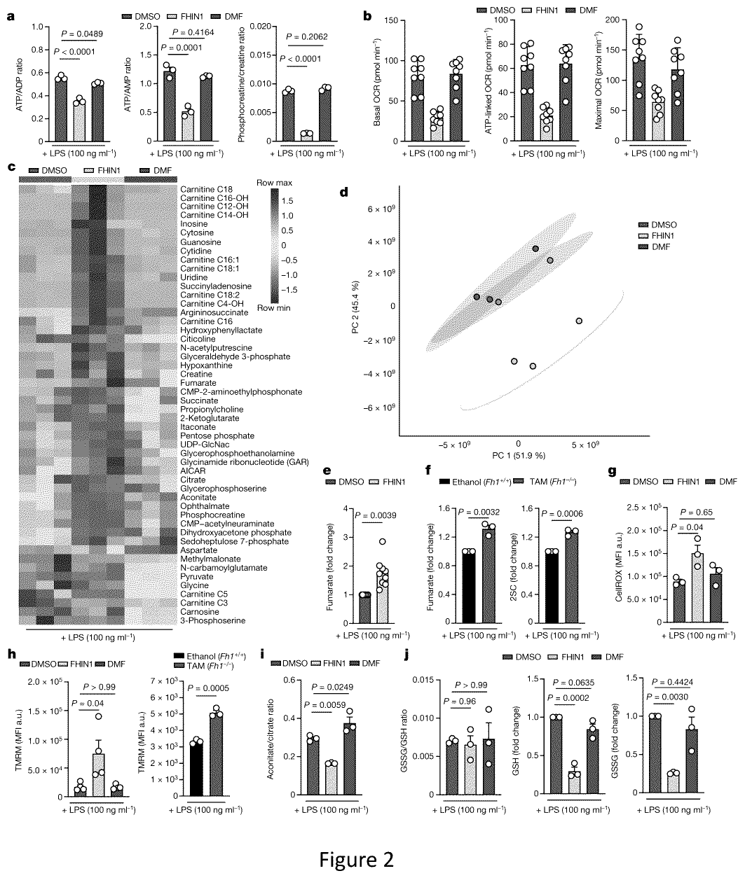

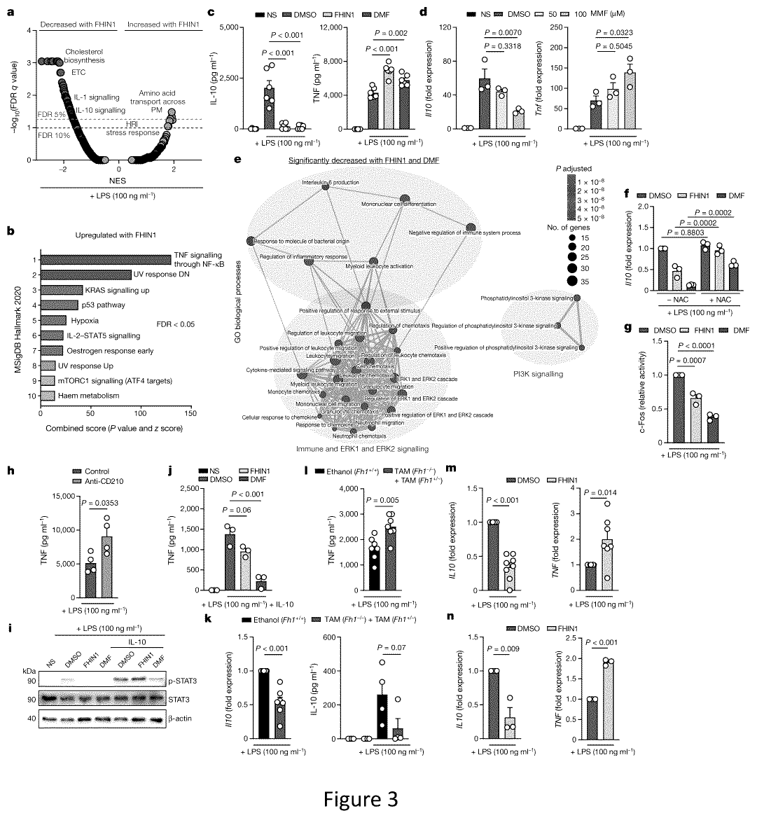

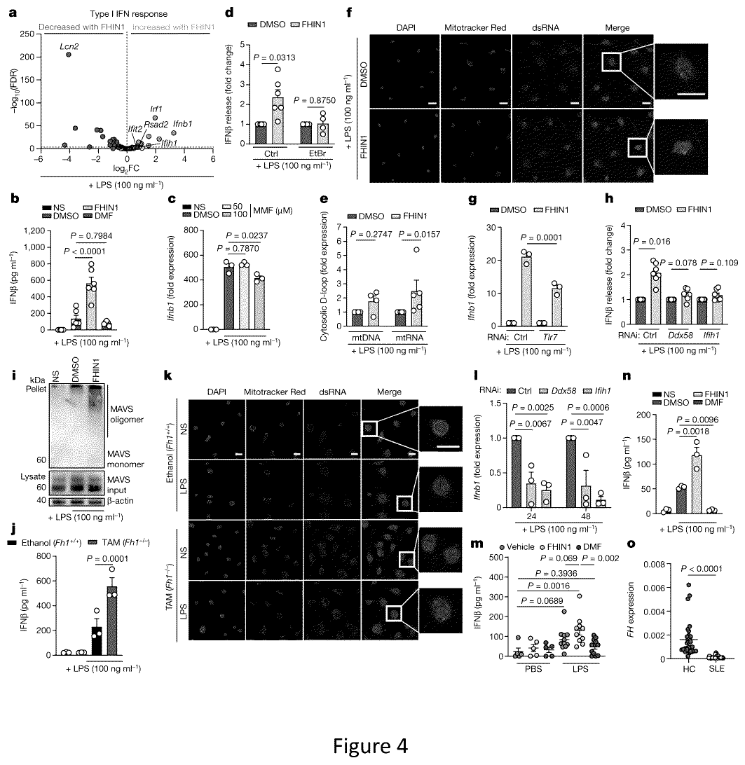

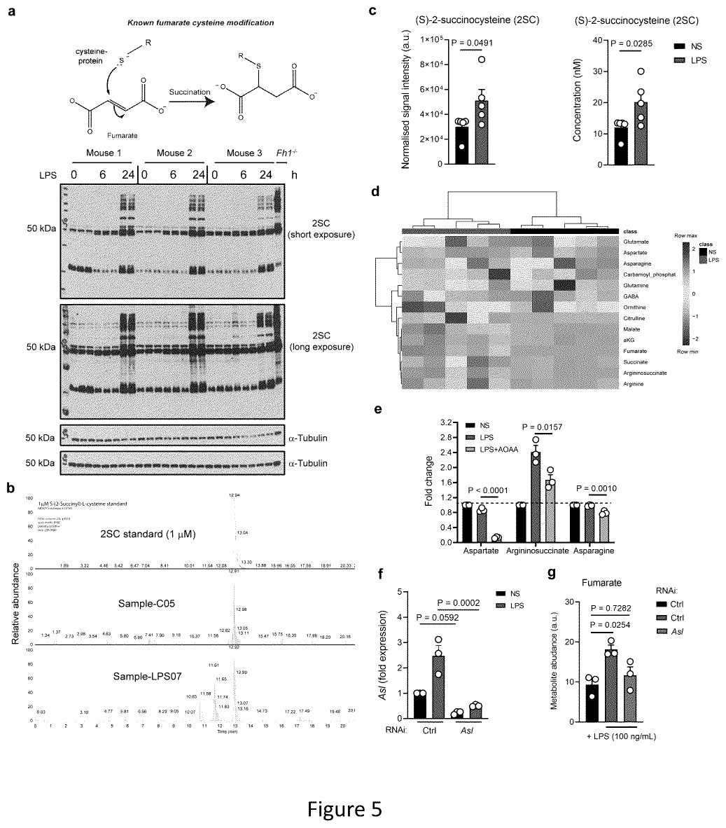

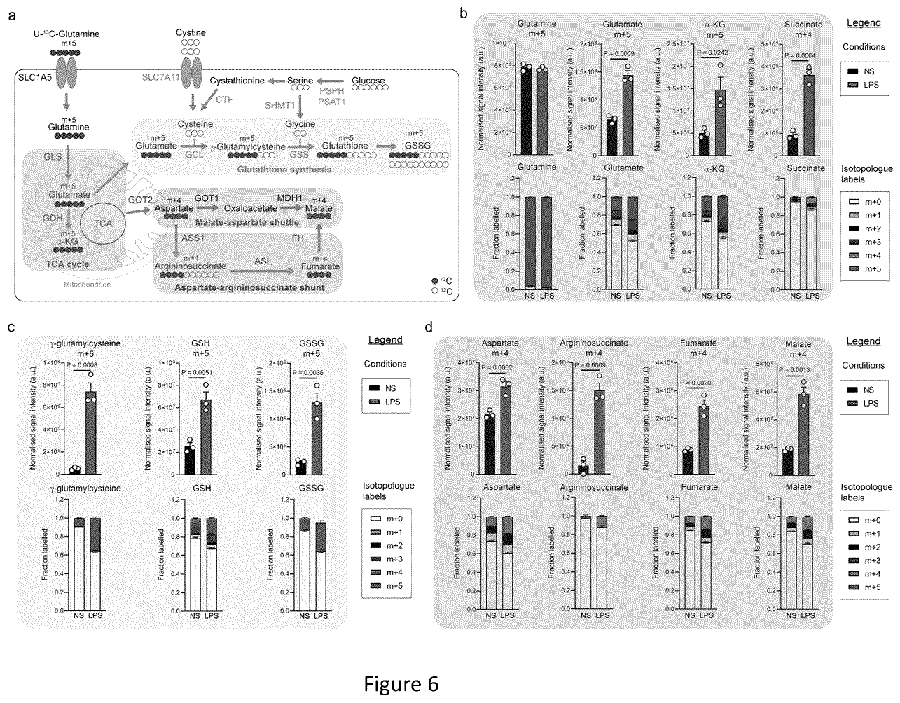

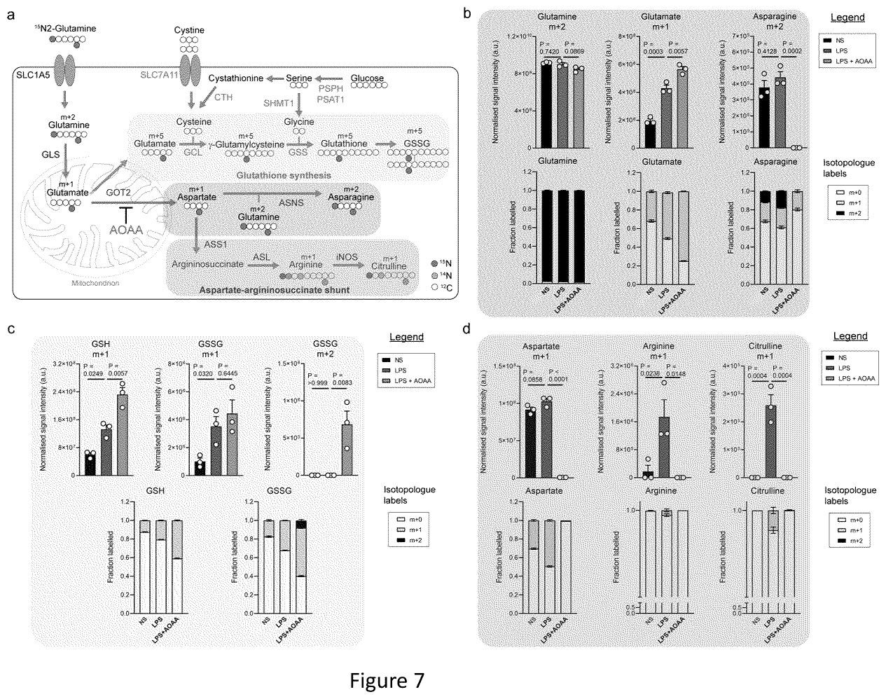

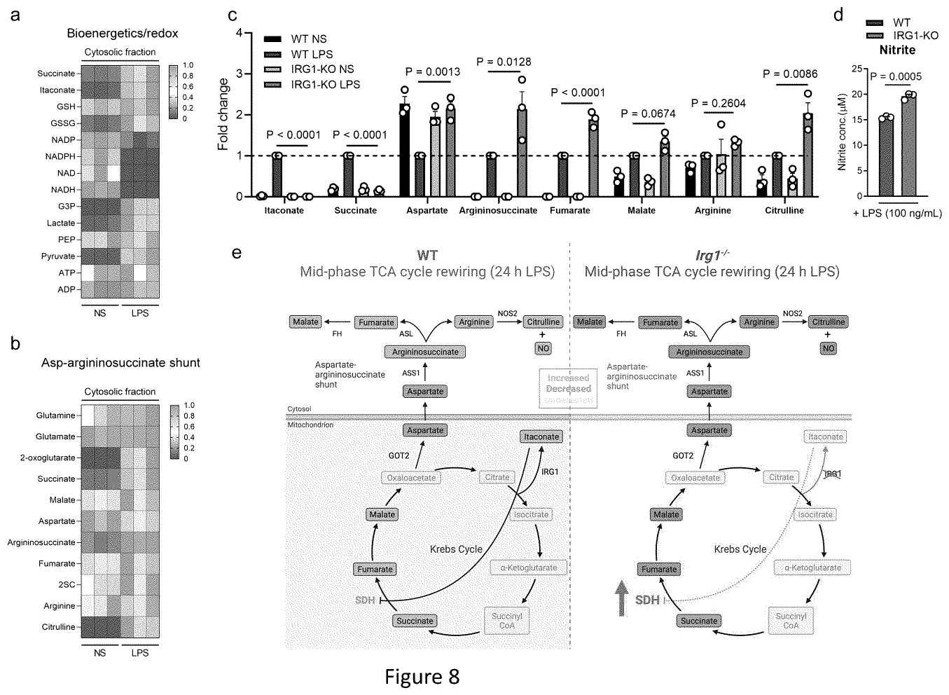

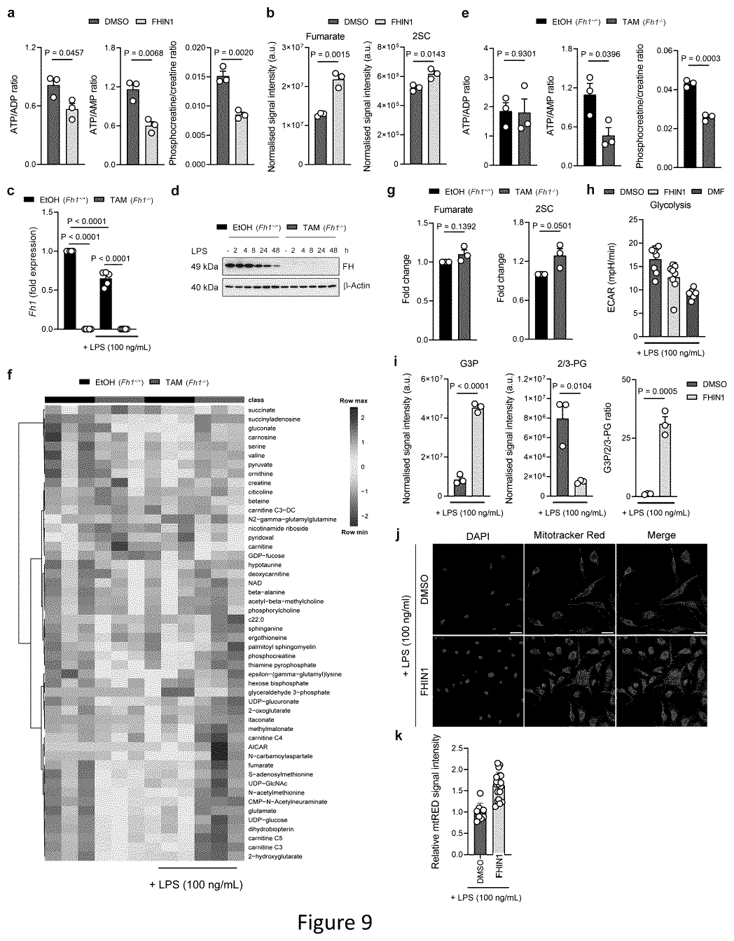

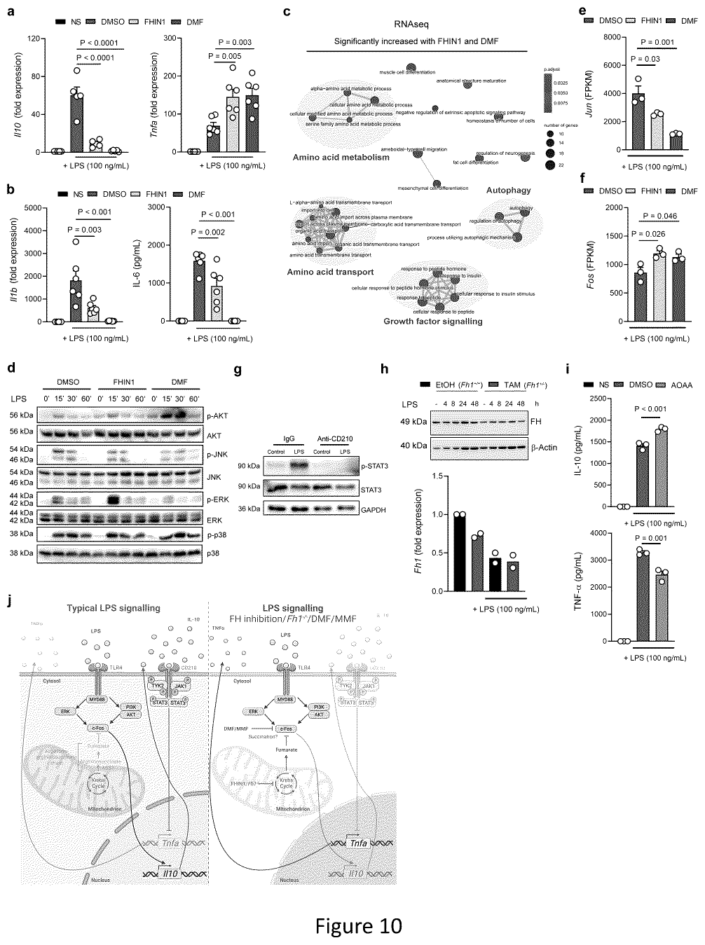

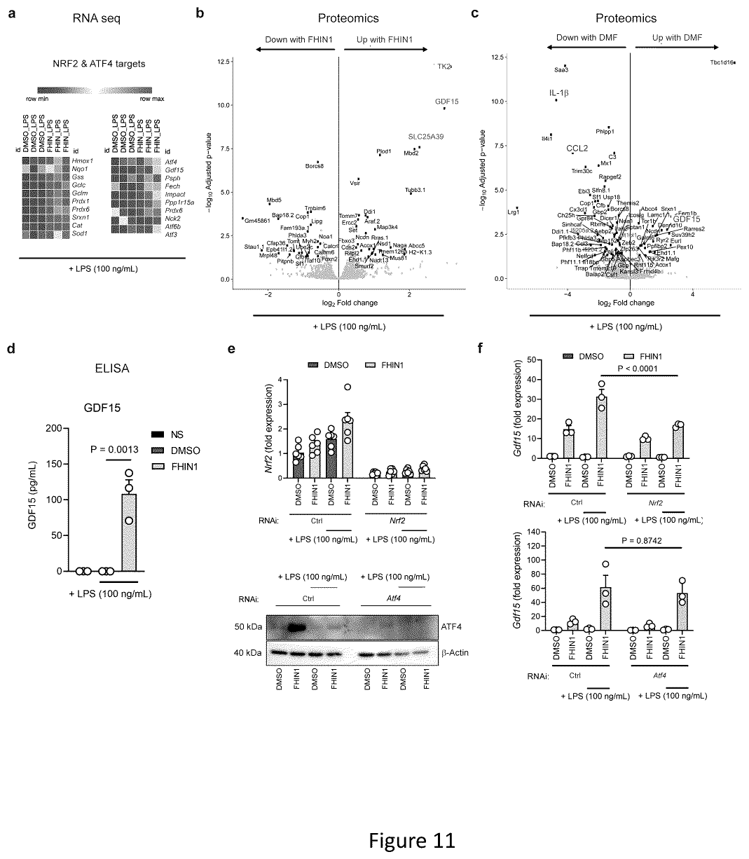

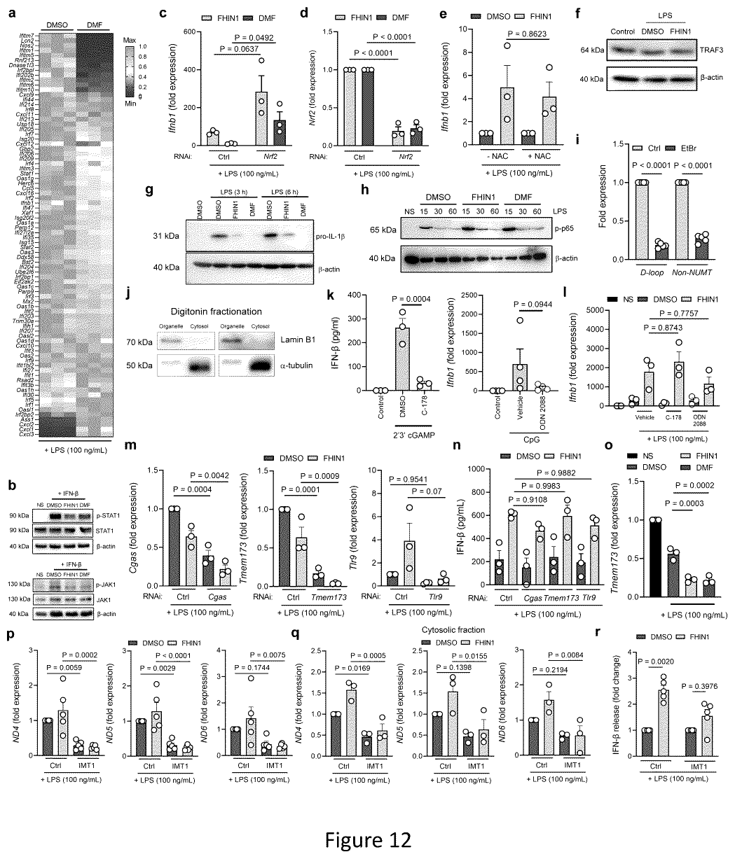

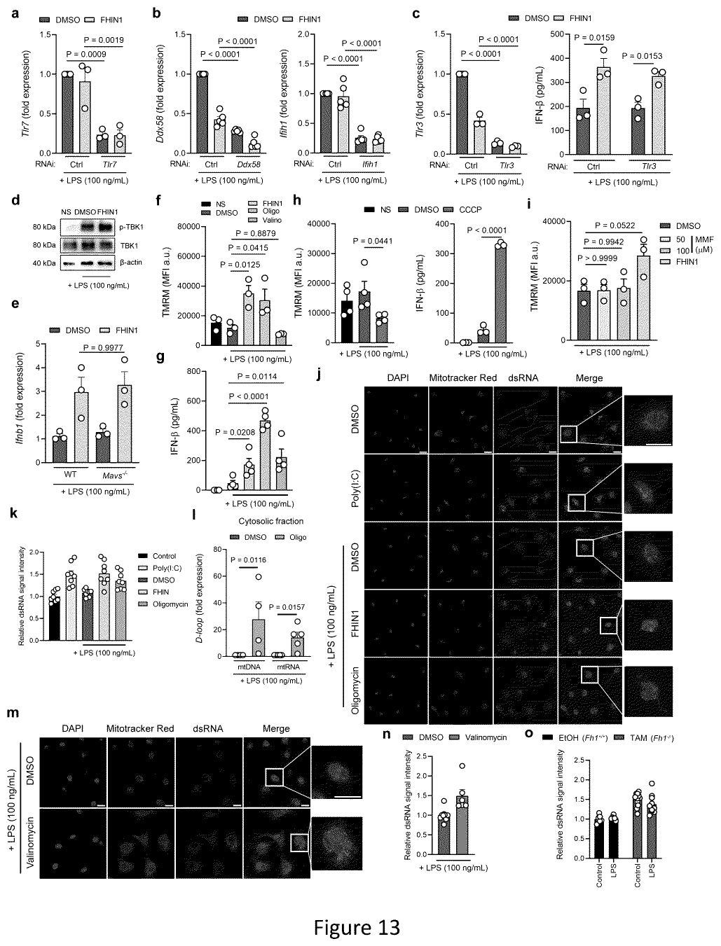

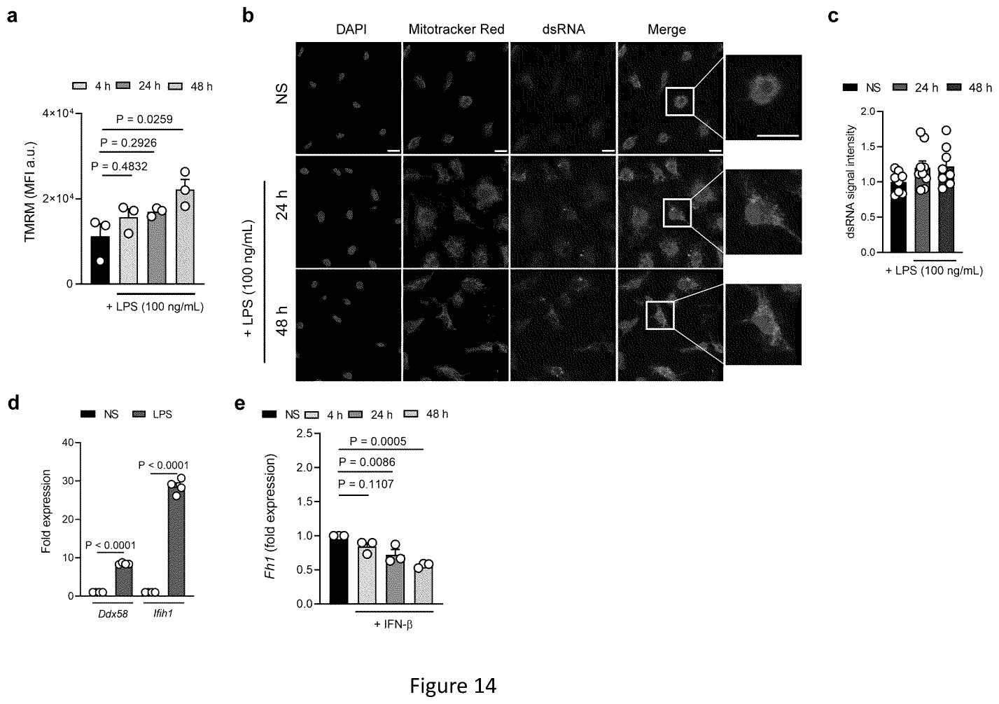

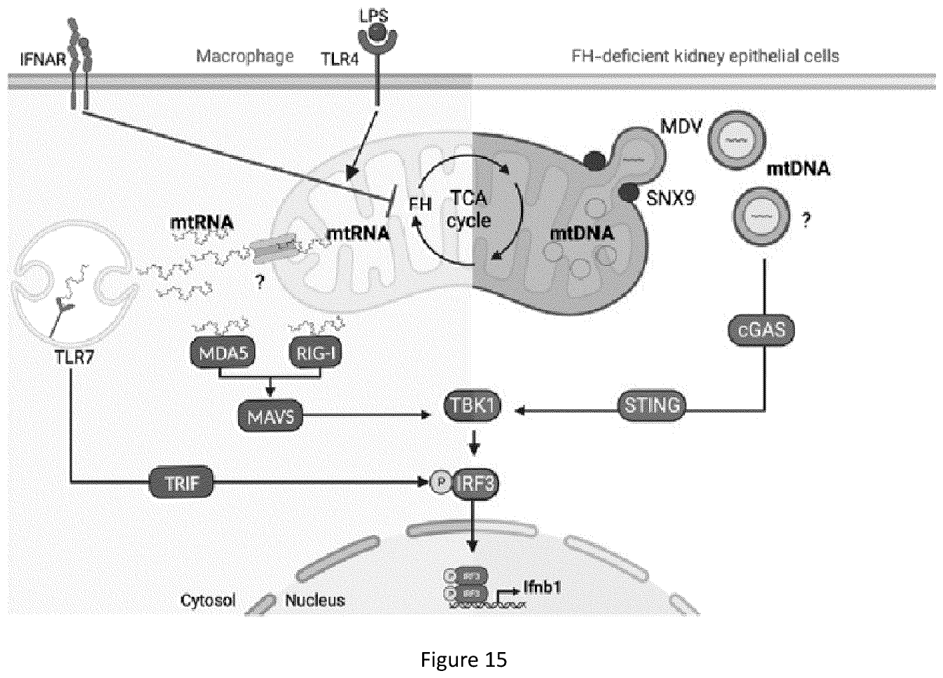

FIG. 5 (Extended Data FIG. 1)—LPS stimulation drives fumarate accumulation and protein succination

a-c, Fumarate-mediated protein succination with LPS (n=3) and 2SC abundance in NS and LPS-stimulated BMDMs (n=5; LPS 4 h). d, Heatmap of metabolites linked to aspartate-argininosuccinate shunt in NS and LPS-stimulated BMDMs (n=5; LPS 24 h) e, Metabolite abundance of aspartate-argininosuccinate shunt metabolites in LPS-stimulated BMDMs pre-treated with DMSO or AOAA (n=3; LPS 4 h; aspartate (P=0.0000005)). f, Asl expression with silencing of Asl following LPS stimulation (n=3; LPS 24 h). g, Fumarate levels with silencing of Asl following LPS stimulation (n=3; LPS 24 h). c,e-h, Data are mean±s.e.m. a, 1 representative blot of 3 shown. n=biological replicates. P values calculated using two-tailed Student's t-test for paired comparisons or one-way ANOVA for multiple comparisons.

FIG. 6 (Extended Data FIG. 2)—LPS stimulation drives fumarate accumulation via glutamine anaplerosis and an aspartate-argininosuccinate shunt

a, Schematic diagram indicating U-¹³C-glutamine tracing into distinct metabolic modules. b, U-¹³C-glutamine tracing into glutamate, α-KG and succinate in LPS-treated BMDMs (m+4 and m+5 labelling intensity and total isotopologue fraction distribution) (n=3; LPS 4 h). c, U-¹³C-glutamine tracing into γ-glutamylcysteine, GSH and GSSG in LPS-treated BMDMs (m+5 labelling intensity and total isotopologue fraction distribution) (n=3; LPS 4 h). d, U-¹³C-glutamine tracing into aspartate, argininosuccinate, fumarate and malate in LPS-treated BMDMs (m+4 labelling intensity and total isotopologue fraction distribution) (n=3; LPS 4 h). Data are mean±s.e.m. n=biological replicates. P values calculated using two-tailed Student's t-test for paired comparisons.

FIG. 7 (Extended Data FIG. 3)—LPS stimulation drives fumarate accumulation via glutamine anaplerosis and an aspartate-argininosuccinate shunt

a, Schematic diagram indicating ¹⁵N₂-glutamine tracing into distinct metabolic modules. b, ¹⁵N₂-glutamine tracing into glutamate and asparagine in LPS-treated BMDMs (m+1 and m+2 labelling intensity and total isotopologue fraction distribution) (n=3; LPS 4 h). c, ¹⁵N₂-glutamine tracing into GSH and GSSG in LPS-treated BMDMs (m+1 and m+2 labelling intensity and total isotopologue fraction distribution) (n=3; LPS 4 h). d, ¹⁵N₂-glutamine tracing into aspartate, arginine and citrulline in LPS-treated BMDMs (m+1 labelling intensity and total isotopologue fraction distribution) (n=3; LPS 4 h; aspartate (P=0.000001)). Data are mean±s.e.m. n=biological replicates. P values calculated using one-way ANOVA for multiple comparisons.

FIG. 8 (Extended Data FIG. 4)—Increase in aspartate-argininosuccinate shunt metabolites in cytosol and Irg1^{/31 /-} macrophages

Heatmap (min-max) of metabolites linked to mitochondrial bioenergetics and redox signalling (a) and the aspartate-argininosuccinate shunt (b) in NS and BMDMs (n=3; LPS 24 h). c, Metabolite abundance of TCA cycle and aspartate-argininosuccinate shunt metabolites in WT and Irg1^{-/-} BMDMs (n=3; LPS 24 h; itaconate (P=0.00000000000002, succinate (P=0.00000003), fumarate (P=0.000018)). d, Nitrite levels in WT and Irg1^{-/-} BMDMs (n=3; LPS 24 h). e, Schematic of metabolic changes occurring during mid-phase TCA cycle rewiring in WT and Irg1^{-/-} BMDMs. Created with BioRender.com. Data are mean±s.e.m. n=biological replicates. P values calculated using two-tailed Student's t-test for paired comparisons or one-way ANOVA for multiple comparisons.

FIG. 9 (Extended Data FIG. 5)—FH deletion increases bioenergetic stress, fumarate, and mitochondrial membrane potential

a, Bioenergetic ratios in BMDMs treated with DMSO or FHIN1 (n=3). b, Fumarate and 2SC levels in BMDMs treated with DMSO or FHIN1 (n=3). qPCR (n=5) (c) and western blot (n=2) (d) analysis of Fh1 expression in Fh1^{+/+} and Fh1^{-/-} BMDMs (EtOH/TAM 72 h; LPS 4 h; Fh1^{+/+} NS vs Fh1^{+/+} LPS (P=0.00000002), Fh1^{+/+} NS vs Fh1^{-/-} NS (P=0.00000000000002), Fh1^{-/-} NS vs Fh1^{-/-} LPS (P=0.0000000000014)). e, Bioenergetic ratios in Fh1^{+/+} and Fh1^{-/-} BMDMs (n=3; EtOH/TAM 48 h). f, Heatmap of top 50 significantly abundant metabolites in Fh1^{+/+} and Fh1^{-/-} BMDMs (n=3; LPS 4 h). g, Fumarate and 2SC levels in Fh1^{+/+} and Fh1^{-/-} BMDMs (n=3; EtOH/TAM 72 h). h, Glycolysis as measured by ECAR in BMDMs pre-treated with DMSO, FHIN1 or DMF (n=8 (DMSO/FHIN1); n=6 (DMF); LPS 4 h). n=technical replicates from 1 experiment performed with 3 pooled biological replicates. Data are mean±s.d. i, Glyceraldehyde 3-phosphate (G3P) and 2,3-phosphoglycerate (2/3-PG) levels and ratio in BMDMs pre-treated with DMSO or FHIN1 (n=3; LPS 4 h; G3P (P=0.00004)). Immunofluorescence (k) and quantification (j) of Mitotracker red staining in BMDMs pre-treated with DMSO or FHIN1 (n=8 (DMSO); n=19 (FHIN1); LPS 4 h). n=technical replicates from representative experiment. Scale bar=20 μm. Data are mean±s.d. a-c,e,g,i Data are mean±s.e.m. Representative blots or images of 2 (d) or 1 experiment(s) (j) shown. n=biological replicates unless stated otherwise. P values calculated using two-tailed Student's t-test for paired comparisons or one-way ANOVA for multiple comparisons.

FIG. 10 (Extended Data FIG. 6)—FH inhibition remodels inflammatory gene expression

a, II10 and Tnfa expression in BMDMs pre-treated with DMSO, FHIN1 or DMF (n=5 (II10); n=6 (Tnfa); LPS 4 h; FHIN1/II10 P=0.000002, DMF/II10 P=0.0000004)). b, II1b expression and IL-6 release in BMDMs pre-treated with DMSO, FHIN1 or DMF (n=6; 4 h LPS; DMF/II1b (P=0.000046), DMF/IL-6 (P=0.00000002)). c, Enrichment map plot of shared significantly increased genes in BMDMs pre-treated with DMF or FHIN1 compared to DMSO control (n=3; LPS 4 h). d, Western blot of total and phospho-AKT, JNK, ERK and p38 levels in BMDMs pre-treated with DMSO, FHIN1 or DMF (n =2). e, Jun expression in RNA seq from BMDMs pre-treated with DMF or FHIN1 compared to DMSO control (n=3; LPS 4 h). f, Fos expression in RNA seq from BMDMs pre-treated with DMF or FHIN1 compared to DMSO control (n=3; LPS 4 h). g, Western blot of total and phospho-STAT3 levels in BMDMs pre-treated with anti-CD210 antibody (1 h) (n=4; LPS 4 h). h, FH protein and gene expression levels in Fh1^{+/+} and Fh1^{+/-} BMDMs (n=2; EtOH/TAM 72 h). i, ELISA of IL-10 and TNF-α release in BMDMs pre-treated with DMSO or AOAA (n=3; LPS 4 h; IL-10 (P=0.000483)). j, Schematic depicting mild suppression of IL-10 expression during typical LPS signalling (right), and increased suppression of IL-10 following FH inhibition, leading to dysregulated TNF-α release (right). Created with BioRender.com. a,b,e,f,h,i Data are mean±s.e.m. 1 representative blot of 2 (d, h) or 4 (g) shown. n=biological replicates. P values calculated using two-tailed Student's t-test for paired comparisons or one-way ANOVA for multiple comparisons.

FIG. 11 (Extended Data FIG. 7)—FH inhibition triggers the NRF2 and ATF4 stress response and promotes GDF15 release

a, Heatmap of significantly differentially expressed RNA seq data in BMDMs pre-treated with FHIN1 compared to DMSO control (n=3; LPS 4 h). Volcano plots of proteomics in BMDMs pre-treated with DMSO, FHIN1 (b) or DMF (c) (n=5; LPS 4 h). d, ELISA of GDF15 in BMDMs pre-treated with DMSO or FHIN1 (n=3; LPS 4 h). e, Nrf2 expression or ATF4 protein levels after silencing of Nrf2 or Atf4, respectively, in BMDMs pre-treated with DMSO or FHIN1 (n=6; LPS 4 h). f, Gdf15 expression after silencing of Nrf2 or Atf4 respectively in BMDMs pre-treated with DMSO or FHIN1 (n=3, LPS 4 h; FHIN1/Nrf2 RNAi (P=0.000048)). d-f, Data are mean±s.e.m. e, 1 representative blot of 6 shown. n=biological replicates unless stated otherwise. P values calculated using one-way ANOVA for multiple comparisons.

FIG. 12 (Extended Data FIG. 8)—IFN-β release following FH inhibition is independent of cGAS-STING

a, Heatmap (min-max) of significantly differentially expressed RNA seq data in BMDMs pre-treated with DMSO or DMF (n=3; LPS 4 h). b, Phospho-STAT1, STAT1, phospho-JAK1 and JAK1 levels in BMDMs pre-treated with DMSO, FHIN1 or DMF (n=3; LPS 4 h). c, Ifnb1 expression after silencing of Nrf2 in BMDMs pre-treated with DMSO, FHIN1 or DMF (n=3, LPS 4 h). d, Nrf2 expression after silencing of Nrf2 in BMDMs pre-treated with DMSO, FHIN1 or DMF (n=3, LPS 4 h; FHIN1 (P=0.0000008), DMF (P=0.0000012)). e, Ifnb1 expression in BMDMs pre-treated with DMSO or FHIN1 in the presence of NAC (n=3; LPS 4 h). f, TRAF3 levels in BMDMs pre-treated with DMSO or FHIN1 (n=3; LPS 4 h). g, IL-1β levels in BMDMs pre-treated with DMSO, FHIN1 or DMF (n=3). h, p-p65 levels in BMDMs pre-treated with DMSO, FHIN1 or DMF (n=3). i, D-loop and Non-NUMT DNA fold expression in EtBr-treated BMDMs (n=5; D-loop (P=000000000031, Non-NUMT (P=0.0000000012). j, Lamin B1 and α-tubulin in cytosolic and membrane-bound organelle fractions following digitonin fractionation (n=3). k, IFN-β release from 2′,3′ cGAMP-or CpG-transfected BMDMs pre-treated (1 h) with C-178 or ODN2088 (n=3 (cGAMP); n=4 (CpG); 3 h). I, Ifnb1 expression in BMDMs pre-treated with DMSO or FHIN1 in conjunction with C-178 or ODN2088 (1 h) respectively (n=3; LPS 4 h). m, Cgas, Tmem173 and Tlr9 expression with silencing of Cgas, Tmem173 and Tlr9 respectively in BMDMs pre-treated with DMSO or FHIN1 (n=3; LPS 4 h). n, IFN-β release with silencing of Cgas, Tmem173 and Tlr9 respectively from BMDMs pre-treated with DMSO or FHIN1 (n=3; LPS 4 h). o, Tmem173 expression in BMDMs pre-treated with DMSO, FHIN1 or DMF (n=3, LPS 4 h). p, ND4, ND5 and ND6 RNA levels in whole cell extracts of BMDMs pre-treated with DMSO or FHIN1 in the presence of IMT1 (n=5; LPS 4 h; ND5 (P=0.000052)). q, ND4, ND5 and ND6 RNA levels in cytosolic extracts of BMDMs pre-treated with DMSO or FHIN1 in the presence or absence of IMT1 (n=5; LPS 4 h). r, IFN-β release in BMDMs pre-treated with DMSO or FHIN1 in the presence of IMT1 (n=3; LPS 4 h). c-e,i,k-r, Data are mean±s.e.m. b,f-h,j, 1 representative of 3 shown. n=biological replicates. P values calculated using two-tailed Student's t-test for paired comparisons or one-way ANOVA for multiple comparisons.

FIG. 13 (Extended Data FIG. 9)—Mitochondrial membrane potential modifiers increase mtRNA and trigger IFN-β release

a, Tlr7 expression with silencing of Tlr7 in BMDMs pre-treated with DMSO or FHIN1 (n=3; LPS 4 h). b, Ddx58 and Ifih1 expression with silencing of Ddx58 and Ifih1 respectively in BMDMs pre-treated with DMSO or FHIN1 (n=5; LPS 4 h; DMSO/Ddx58 (P=0.000000000002), FHIN1/Ddx58 (P=0.000000813792), DMSO/Ifih1 (P=0.00000009), FHIN1//fih1 (P=0.00000014)). c, Tlr3 expression and IFN-β release with silencing of Tlr3 in BMDMs pre-treated with DMSO or FHIN1 (n=3; LPS 4 h; DMSO/Tlr3 (P=0.000000007), FHIN1/Tlr3 (P=0.000013487)). d, TBK1 and p-TBK1 in BMDMs pre-treated with DMSO or FHIN1 (n=3; LPS 4 h). e, Ifnb1 expression in WT and Mavs^{-/-} BMDMs pre-treated with DMSO or FHIN1 (n=3; LPS 4 h). f, MFI of TMRM staining in BMDMs pre-treated with DMSO, FHIN1, oligomycin or valinomycin (n=3, LPS 4 h). g, IFN-β release from BMDMs pre-treated with DMSO, FHIN1, oligomycin or valinomycin (n=4; LPS 4 h; oligomycin (P=0.0000003)). h, MFI of TMRM staining and IFN-B release from BMDMs pre-treated with DMSO or CCCP (n=4 (TMRM), n=3 (IFN-B); LPS 4 h; CCCP/IFN-B (P=0.00000008). i, MFI of TMRM staining in BMDMs pre-treated with DMSO or MMF (n=3, LPS 4 h). Immunofluorescence (j) and quantification (k) of dsRNA in BMDMs pre-treated with DMSO, FHIN1 or oligomycin or transfected with poly (I:C) (n=8; LPS 4 h). n=technical replicates from representative experiment. Data are mean±s.d. Scale bar=20 μm. I, D-loop fold expression in DNA and RNA isolated from cytosolic fractions of digitonin-fractionated BMDMs pre-treated with DMSO or oligomycin (n=4 for mtDNA, n=5 for mtRNA). Immunofluorescence (m) and quantification (n) of dsRNA in BMDMs pre-treated with DMSO or valinomycin (n=9 (DMSO); n=6 (Valinomycin); LPS 4 h). n=technical replicates from representative experiment. Data are mean±s.d. Scale bar=20 μm. o, Quantification of dsRNA immunofluorescence in Fh1^{+/+} and Fh1^{-/-} BMDMs (n=7 (Fh1^{+/+} Control); n=6 (Fh1^{+/+} LPS); n=12 (Fh1^{-/-} Control); n=10 (Fh1^{-/-} LPS); EtOH/TAM 72 h; LPS 4 h). n=technical replicates from representative experiment. Data are mean±s.d. a-c,e-i,l Data are mean±s.e.m. d,j,m, 1 representative blot or image of 3 experiments shown. n=biological replicates unless stated otherwise. P values calculated using two-tailed Student's t-test for paired comparisons, one-way ANOVA for multiple comparisons.

FIG. 14 (Extended Data FIG. 10)—Prolonged LPS stimulation increases mitochondrial membrane potential and dsRNA

a, MFI of TMRM staining in BMDMs (n=3). Immunofluorescence (b) and quantification (c) of dsRNA in BMDMs (n=8 (0/48 h); n=9 (24 h)). n=technical replicates from representative experiment. Data are mean±s.d. Scale bar=20 μm. d, Ddx58 and Ifih1 expression in BMDMs (n=4; LPS 4 h; Ddx58 (P=0.0000000010), Ifih1 (P=0.00000012)). e, Fh1 expression in IFN-β-stimulated BMDMs (n=3). a,d,e, Data are mean±s.e.m. b, 1 representative image of 3 experiments shown. n=biological replicates unless stated otherwise. P values calculated using two-tailed Student's t-test for paired comparisons, one-way ANOVA for multiple comparisons.

FIG. 15 illustrates that FH loss drives a type I IFN response. FH loss in both macrophages (left) and kidney epithelial cells (right) has been found to drive a type I IFN response through the release of mitochondrial nucleic acids. In macrophages, mtRNA activates cellular RNA sensors while in kidney epithelial cells mtDNA exits mitochondria in vesicles which activates the cGAS-STING pathway. Thus, in macrophages, FH impairment does not signal through DNA sensing pathways and primarily activates both dsRNA sensing via RIG-I and MDA5 as well as ssRNA sensing via Toll-like receptor 7 (TLR7). Abbreviations: cGAS, cyclic GMP-AMP synthase; FH, fumarate hydratase; IL-10, interleukin-10; LPS, lipopolysaccharide; NF-κB, nuclear factor-κB; TCA, tricarboxylic acid; TLR4, Toll-like receptor 4; TNF, tumour necrosis factor; TRIF, TIR domain-containing adaptor inducing interferon-β.

### DETAILED DESCRIPTION

The present invention focuses on the fumarate hydratase metabolic pathway and its impact on the immune response, specifically the innate immune response.

As discussed in Hooftman, A., Peace, C. G., Ryan, D. G. et al. Macrophage fumarate hydratase restrains mtRNA-mediated interferon production. Nature 615, 490-498 (2023); and Christian G. Peace, Shane M. O'Carroll, and Luke A. J. O'Neill, Fumarate hydratase as a metabolic regulator of Immunity, Trends in Cell Biology, June 2024, Vol. 34, No. 6), our findings demonstrate that fumarate has specific roles in macrophage activation, regulating the production of such cytokines as interleukin (IL)-10 and type I interferons (IFNs). It has also been shown that that FH loss leads to mitochondrial nucleic acid release (i.e. DNA and RNA release from mitochondria) which are sensed by cytosolic nucleic acid (CAN) sensors including retinoic acid-inducible gene (RIG)-I, melanoma differentiation-associated protein (MDA)5, and cyclic GMP-AMP synthase (cGAS) to upregulate interferon, specifically IFN-β, production. Furthermore, as FH was expressed at low levels in patients with SLE, this suggests that mitochondrial nucleic acids may be pathological in this autoimmune disease. We conclude that methods preventing these signalling pathways (e.g., by blocking MDA-5 etc) may be useful in treatment. Although type I IFN signalling is already a target in systemic lupus erythematosus (SLE), it could be plausible that pharmacological inhibition mitochondrial transcription with the recently developed POLRMT inhibitor, IMT1, could prevent the induction of type I IFN responses in these patients. Taken together, all these findings have relevance in the pathogenesis and treatment of diseases associated with decreased FH levels such as systemic lupus erythematosus (SLE) or FH-deficient kidney cancer.

In a general context, the invention identifies and provides a new therapeutic target and relates to an anti-inflammatory agent for use in upregulating fumarate hydratase activity and/or expression to reduce or prevent inflammation, specifically interferon production. The anti-inflammatory agent is a fumarate hydratase modulating agent, which may include fumarate hydratase itself, as discussed below.

A general embodiment of the invention relates to a method for the prevention and/or treatment of inflammation, including interferon driven inflammation, in a patient in need thereof, the method comprising administering an effective amount of a fumarate hydratase modulating agent, which may include administering fumarate hydratase itself, to treat said patient.

The patients in need to treatment are those patients with decreased FH activity/expression, specifically those with interferon-driven diseases that have decreased FH activity/expression.

Typically said patient has low levels of fumarate hydratase expression compared to a healthy subject. For example, in patients with systemic lupus erythematosus (SLE). fumarate hydratase levels (FH) are significantly suppressed compared to healthy subjects and levels of FH were on average undetectable in the SLE patients.

Typically, said patient has an interferon-driven disease associated with decreased FH activity and/or expression.

Accordingly, we have identified a new protective role for FH in the innate immune response and inflammation, specifically in maintaining appropriate macrophage, cytokine and/or interferon responses. Specifically, the invention focuses on utilising these findings to suppress the immune system and/or reduce pro-inflammatory responses in a patient in need thereof. These findings provide a mechanism for suppressing any undesired or overactive immune response in the patient.

The general objective of this treatment is to upregulate FH activity and/or expression. Ideally, the objective is to restore fumarate hydratase to normal levels. It will be understood that the fumarate hydratase modulating agent upregulates fumarate hydratase activity and/or expression to reduce or prevent macrophage, cytokine and/or interferon production in a patient in need thereof.

There are currently very few therapeutic strategies which upregulate (in contrast to inhibiting or impairing) metabolic pathways. This upregulation strategy is advantageous as it is expected that there would be very few off-target and unwanted side effects, as the objective is simply to maintain the cellular FH metabolism.

A specific embodiment of the invention relates to a method for the prevention and/or treatment of interferon driven inflammation. In this manner, the objective is to upregulate fumarate hydratase activity and/or expression to reduce or prevent aberrant or unwanted type 1 interferon (type 1 IFN), specifically IFN-β, production in a patient in need thereof.

Alternatively, or additionally, the objective is to upregulates fumarate hydratase activity and/or expression to reduce or prevent interleukin-10 (IL-10) and/or tumor necrosis factor (TNF) production in a patient in need thereof.

In this manner, one of the general objectives of this method is to upregulate and/or restore fumarate hydratase levels in order to prevent downstream RIG-I/MDA5 and TLR7 triggering the release of cytokines. By restoring upstream fumarate hydratase levels, we have found that this blocks the release of mitochondrial RNA that is being sensed by MDA5 and TLR. These findings highlight the importance of mitochondrial control of innate immune responses including how mitochondrial dysregulation contributes to interferon-driven diseases.

The therapeutic targeting of type I IFN signalling however has been a very popular therapeutic target in inflammation with the development of JAK inhibitors (e.g. baracitinib) to prevent downstream IFN signalling and anti-IFNAR monoclonal antibodies to impair IFN binding to its receptor. In contrast, our findings relate to a new upstream therapeutic target and the objectives outlined above are achieved by administering a FH modulating agent, which may include administering fumarate hydratase itself.

The method of the invention may comprise administering an effective amount of a fumarate hydratase modulating agent, which may include administering fumarate hydratase itself, to treat said patient.

It will be understood that the fumarate hydratase modulating agent is any agent which modulates fumarate hydratase levels and/or expression in said patient.

Ideally, said fumarate hydratase modulating agent is selected from one or more of the following:
fumarate hydratase;
a small molecule activator of fumarate hydratase;
a pharmaceutical composition comprising fumarate hydratase or a small molecule activator of fumarate hydratase;
a viral vector comprising a fumarate hydratase gene or mRNA thereof; or
a non-viral vector comprising a fumarate hydratase gene or mRNA thereof.

### Protein/Enzyme Replacement Therapy—Fumarate Hydratase

Fumarate hydratase (FH) is an enzyme of the Tricarboxylic Acid (TCA) cycle whose mutations lead to hereditary and sporadic forms of cancer. FH is a highly conserved homotetrameric cytosolic and mitochondrial enzyme expressed in most tissues, except the ovaries, vagina, muscle, adipose tissue, and bone marrow. The same transcript encodes both forms, and the localization is determined by the cleavage of the N-terminal mitochondrial targeting sequence. In the cytosol, FH participates in pathways where fumarate is produced, such as the urea cycle and the purine nucleotide cycle (PNC). In the mitochondria, FH catalyses the reversible hydration of fumarate to malate as a step of the tricarboxylic acid cycle (TCA) cycle, also known as the Krebs cycle or citric acid cycle. FH inactivation leads to aberrant accumulation of fumarate, affecting this essential pathway and thus the production of cellular energy and generation of macro-molecular precursors.

The review article Peace C G, O'Carroll S M, O'Neill L A J. Fumarate hydratase as a metabolic regulator of immunity. Trends Cell Biol. 2024 June; 34 (6):442-450 is a review of FH as a metabolic regulator of immunity, is a review paper on FH.

According to one embodiment, fumarate hydratase itself or a recombinant protein thereof may be administered to modulate fumarate hydratase levels in a patient.

The present invention also further provides a pharmaceutical composition comprising fumarate hydratase itself or a recombinant protein thereof, and optionally one or more pharmaceutically acceptable excipient(s).

Conventional means for administering the protein/enzyme therapy may be used, which preferably include the use of a vector, such as a nanoparticle for example.

Alternatively, fumarate hydratase itself or a recombinant protein thereof or pharmaceutical composition may be administered by any means that achieves the generally intended purpose.

Non-limiting acceptable means of administration include oral and parenteral routes. Ideally, the fumarate hydratase itself or a recombinant protein thereof or pharmaceutical composition is administered orally. Alternatively, administration may be accomplished parentally. Methods of parenteral delivery include topical, intra-arterial, intramuscular, subcutaneous, intramedullary, intrathecal, intraventricular, intravenous, intraperitoneal, intrauterine, intravaginal, sublingual or intranasal administration.

The dosage administered will depend on the age, health and weight of the recipient, the type of concurrent treatment, if present, the frequency of treatment, and the nature of the desired effect. Although individual needs may vary from patient to patient, it is within the ability of a clinician of ordinary skill to determine the optimal range for an effective amount of all ingredients.

The pharmaceutical composition may be formulated as an aqueous solution, including a suspension.

The fumarate hydratase itself or a recombinant protein thereof may also be administered as part of a combination therapy with other agents.

### Small Molecule Therapy—Small Molecule Activators Of Fumarate Hydratase

Any small molecule agent which modulates FH expression may be used. For example, a series of phenyl-pyrrolo-pyrimidine-diones were identified as activators of human fumarate hydratase in Zhu H et al, Identification of Activators of Human Fumarate Hydratase by Quantitative High-Throughput Screening. SLAS Discov. 2020 January; 25(1):43-56.

The present invention also further provides a pharmaceutical composition comprising small molecule activator of fumarate hydratase, and optionally one or more pharmaceutically acceptable excipient(s).

This small molecule activator or pharmaceutical composition may be administered by any means that achieves the generally intended purpose.

Non-limiting acceptable means of administration include oral and parenteral routes.

Ideally, the small molecule activator or pharmaceutical composition is administered orally. Alternatively, administration may be accomplished parentally. Methods of parenteral delivery include topical, intra-arterial, intramuscular, subcutaneous, intramedullary, intrathecal, intraventricular, intravenous, intraperitoneal, intrauterine, intravaginal, sublingual or intranasal administration.

The dosage administered will depend on the age, health and weight of the recipient, the type of concurrent treatment, if present, the frequency of treatment, and the nature of the desired effect. Although individual needs may vary from patient to patient, it is within the ability of a clinician of ordinary skill to determine the optimal range for an effective amount of all ingredients.

The pharmaceutical composition may be formulated as an aqueous solution, including a suspension.

The activator may also be administered as part of a combination therapy with other agents.

### Gene Therapy—A Viral Or Non-Viral Vector Comprising A Fumarate Hydratase Gene Or Mrna Thereof

### A) FH Gene/Protein Sequence

The FH gene encodes the protein fumarate hydratase (FH), a key enzyme in the tricarboxylic acid (TCA) cycle.

FH is located on chromosome 1 (1q42.3-q43) and is composed of 10 exons. The FH protein is a 510-amino acid structure with three key domains: a N-terminal lyase 1 domain, a C-terminal fumarase C domain and a central domain which interacts with other FH monomers. In its functional form, FH exists as a homotetramer and can be localised in the mitochondria or the cytosol. Both mitochondrial and cytosolic FH are encoded by nuclear DNA, with the differential localisation mediated by alternative transcription products, one of which produces a precursor protein with an N-terminal mitochondrial localisation signal. The protein sequence is provided below

| ORIGIN | |
|---|---|
| 1 | myralrllar srplvrapaa alasapglgg aavpsfwppn aarmasqnsf rieydtfgel |
| 61 | kvpndkyyga qtvrstmnfk iggvtermpt pvikafgilk raaaevnqdy gldpkianai |
| 121 | mkaadevaeg klndhfplvv wqtgsgtqtn mnvnevisnr aiemlggelg skipvhpndh |
| 181 | vnksqssndt fptamhiaaa ievhevllpg lqklhdalda kskefaqiik igrthtqdav |
| 241 | pltlgqefsg yvqqvkyamt rikaampriy elaaggtavg tglntrigfa ekvaakvaal |
| 301 | tglpfvtapn kfealaahda lvelsgamnt tacslmkian dirflgsgpr sglgelilpe |
| 361 | nepgssimpg kvnptqceam tmvaaqvmgn hvavtvggsn ghfelnvfkp mmiknvlhsa |
| 421 | rllgdasvsf tencvvgiga nterinklmn eslmlvtaln phigydkaak iaktahkngs |
| 481 | tlketaielg yltaeqfdew vkpkdmlgpk |

Source: https://www.ncbi.nlm.nih.gov/protein/NP_000134.2 This sequence is incorporated herein in its entirety.

The DNA sequence (29152 bp) is provided here:

https://www.ncbi.nlm.nih.gov/nuccore/241666449 This sequence is incorporated herein in its entirety.

### B) A Viral Vector Comprising a Fumarate Hydratase Gene or Mrna Thereof

The fumarate hydratase modulating agent may be delivered using conventional gene therapy viral vector approaches. Viral vector gene therapies use engineered viral delivery systems to introduce a FH gene nucleic acid or mRNA thereof into cells.

Vectors include lentiviral vectors, Adenoviral vectors, Adeno-associated vectors (AAV).

The review article, Di Donfrancesco A, et al, Gene Therapy for Mitochondrial Diseases: Current Status and Future Perspective. Pharmaceutics. 2022 Jun. 17; 14(6):1287 Gene Therapy for Mitochondrial Diseases: Current Status and Future Perspective—PMC (nih.gov), discusses recent approaches which may be used.

### C) A Non-Viral Vector Comprising a Fumarate Hydratase Gene or mRNA Thereof

The fumarate hydratase modulating agent may also be delivered using a non-viral gene therapy approach as discussed in Di Donfrancesco A, et al, Gene Therapy for Mitochondrial Diseases: Current Status and Future Perspective. Pharmaceutics. 2022 Jun. 17; 14(6):1287.

These approaches are well known to the skilled person and include physical methods such as the hydrodynamic or ballistic injection of DNA and chemical methods include micelles of cationic surfactants, rhodamine nanoparticles, and liposomes. Cationic liposomes, consisting of microscopic particles with mono- or multi-cationic head groups, are the most used method for gene delivery.

Lipid nanoparticles may be advantageously used for delivery of the fumarate hydratase gene or mRNA thereof. Michaela Jeong, et al, Lipid nanoparticles (LNPs) for in vivo RNA delivery and their breakthrough technology for future applications, Advanced Drug Delivery Reviews, Volume 200, 2023, 114990, discusses recent approaches to lipid nanoparticle delivery.

RNA therapeutics may also be used comprising the delivery of synthetic mRNA to result in exogenous protein expression. LNPs enable the efficient delivery of RNA molecules into target cells and tissues.

Ideally, the method comprises the delivery of fumarate hydratase itself or the fumarate hydratase modulating agent, such as a small molecule activator of fumarate hydratase or the fumarate hydratase gene or mRNA thereof, to inflammatory cells or pro-inflammatory cells (including macrophages) of said patient.

Optionally, the method is for the treatment of inflammation of the skin, kidneys and/or joints in said patient. This includes the treatment of auto-immune diseases such as systemic lupus erythematosus (SLE), asthma, arthritis, sepsis and covid-19.

Advantageously, the method may be used for the treatment of chronic inflammation in said patient.

Still optionally, the method may be used for the treatment of sepsis during bacterial or viral infections in said patient.

Another embodiment of the invention is fumarate hydratase for use in the prevention and/or treatment of inflammation, preferably interferon driven inflammation.

A further embodiment of the invention is an anti-inflammatory agent for use in upregulating fumarate hydratase activity and expression to reduce or prevent interferon production, wherein the anti-inflammatory agent is
fumarase hydratase;
a small molecule activator of fumarate hydratase;
a pharmaceutical composition comprising fumarate hydratase or said small molecule activator of fumarate hydratase;
a viral vector comprising a fumarate hydratase gene or mRNA thereof; or
a non-viral vector, such as a lipid nanoparticle, comprising a fumarate hydratase gene or mRNA thereof.

Stimulation of macrophages with the TLR4 ligand lipopolysaccharide (LPS) induces metabolic reprogramming involving rewiring of the TCA cycle and mitochondrial respiration, facilitating cytokine production. Changes in macrophage metabolism have emerged as a major regulator of inflammation^{2,4-6}. While metabolic reprogramming is crucial for macrophage activation⁷, the players involved and how they regulate cytokine production remain incompletely characterised.

### Accumulation of Fumarate in Macrophages

To evaluate metabolic alterations that occur during LPS stimulation, we employed an unbiased liquid chromatography-mass spectrometry (LC-MS)-based metabolomics approach to characterise the metabolome of inflammatory bone marrow-derived macrophages (BMDMs). The TCA cycle metabolite fumarate stood out as one of the most significantly upregulated metabolites upon exposure to acute LPS stimulation, joining previously identified metabolites such as itaconate² (FIG. 1a). We also observed a significant increase in fumarate-mediated protein succination⁸⁻¹⁰, resulting in the formation of the fumarate-cysteine adduct, (S)-2-succinocysteine (2SC) (FIG. 5a-c-Extended Data FIG. 1a-c).

As acute LPS stimulation failed to impair respiration (FIG. 1b, c), TCA cycle disruption is unlikely to be sufficient for fumarate accumulation. Increased flux through the aspartate-argininosuccinate shunt has been reported to support nitric oxide (NO) production⁵. As fumarate is a by-product of argininosuccinate cleavage by argininosuccinate lyase (ASL) in the cytosol, we hypothesised that argininosuccinate may be a source of fumarate. Supporting this, we observed decreased aspartate, the substrate for argininosuccinate, and increased argininosuccinate, fumarate, and malate levels (FIG. 1d), consistent with increased flux through the shunt. This rewiring also occurred during prolonged LPS stimulation (FIG. 5d-Extended Data FIG. 1d).

Argininosuccinate synthase (Ass1) and fumarate hydratase (Fh1) expression increased and decreased respectively in LPS-stimulated BMDMs, as determined by RT-qPCR (FIG. 1e). Using available quantitative proteomics data^{2,11}, we found argininosuccinate synthase (ASS1) to be upregulated, whereas levels of glutamic-oxaloacetic transaminase 2 (GOT2), ASL and FH were not significantly altered (FIG. 1f). FH protein levels were suppressed only at later time points of LPS (FIG. 1g), indicating that ASS1 induction is vital to the acute accumulation of fumarate.

Inhibition of the aspartate-argininosuccinate shunt with the GOT2 inhibitor aminooxyacetic acid (AOAA) 5 reduced aspartate, asparagine, argininosuccinate and fumarate levels following LPS stimulation (FIG. 1h and FIG. 5e-Extended Data FIG. 1e). Knockdown of Asl also prevented fumarate accumulation (FIG. 5f-Extended Data FIG. 1f, g) indicating its dependency on the aspartate-argininosuccinate shunt, which would increase cytosolic fumarate (FIG. 1i). With stable isotope-assisted tracing, we show that glutamine-dependent anaplerosis is in part responsible for fumarate accumulation and drives the aspartate-argininosuccinate shunt. U-¹³C-glutamine tracing demonstrated glutaminolysis as a carbon source for the TCA cycle, aspartate-argininosuccinate shunt metabolites, including fumarate, and glutathione (FIG. 6-Extended Data FIG. 2). ¹⁵N₂-glutamine tracing also demonstrated that glutamine nitrogen is a source for glutathione synthesis and aspartate-argininosuccinate shunt metabolites (FIG. 7-Extended Data FIG. 3). Importantly, AOAA completely prevented the contribution of glutamine nitrogen to aspartate, asparagine, arginine and citrulline, confirming its inhibition of GOT2. Metabolomics on cytosolic fractions of resting and LPS-stimulated macrophages showed that metabolites such as itaconate and succinate accumulate in the cytosol following LPS stimulation (FIG. 8a-Extended Data FIG. 4a). Importantly, we also found increased cytosolic argininosuccinate, fumarate and 2SC (FIG. 8b-Extended Data FIG. 4b).

We hypothesised that Irg1^{-/-} BMDMs (which are unable to synthesise itaconate) would relieve inhibition of succinate dehydrogenase (SDH)^{4,12} and exhibit greater accumulation of aspartate-argininosuccinate shunt metabolites. Metabolomics in Irg1^{-/-} BMDMs revealed the expected decrease in itaconate and succinate, and increased aspartate-argininosuccinate shunt metabolites, including fumarate and NO (FIG. 8c,d-Extended Data FIG. 4c, d), providing further evidence linking mitochondrial TCA cycle activity to an aspartate-argininosuccinate shunt (FIG. 8e-Extended Data FIG. 4e).

### FH Inhibition Causes Metabolic Rewiring

FH catalyses the hydration of fumarate to malate in the mitochondrion and cytosol¹³, the inhibition of which elevates cytosolic fumarate accumulation, perturbs urea cycle metabolism and leads to renal cyst development¹⁴. Given protein levels of FH remain stable during early LPS stimulation (FIG. 1g), we used a well-established pharmacological inhibitor of FH (FHIN1)¹⁵ and a recently developed tamoxifen-inducible CRE-ERT2-Fh1^{-/-} model to probe the role of FH activity and fumarate accumulation in macrophages. However, since FH inhibition may lead to effects independent of fumarate accumulation through mitochondrial and redox stress¹⁶, we also used low concentrations of cell-permeable dimethyl fumarate (DMF) to deliver a cysteine-reactive fumarate ester which does not inhibit respiration¹⁷⁻¹⁹. This approach would uncouple the role of impaired mitochondrial bioenergetics following TCA cycle disruption and fumarate-mediated electrophilic modification of cysteine residues.

Previous reports show that immunometabolites and their derivatives affect macrophage function through regulation of metabolic pathways^{9,20,21}. We therefore aimed to assess how FH inhibition and DMF may regulate macrophage metabolism. First, comparing the effects of FHIN1 and DMF on mitochondrial bioenergetics, we found that FHIN1 reduced ratios of ATP/ADP, ATP/AMP, and P-creatine/creatine while DMF had no effect, demonstrating that FH sustains mitochondrial bioenergetics (FIG. 2a, FIG. 9a-Extended Data FIG. 5a). This was confirmed by respirometry, showing FHIN1 impaired basal respiration, ATP production and maximal respiration as measured by OCR, while DMF had no effect (FIG. 2b). FHIN1 led to a distinct metabolic signature characterised by alterations in TCA cycle metabolites including citrate, aconitate, itaconate and succinate, indicating TCA cycle rewiring, as well as enhanced fumarate and 2SC accumulation, supporting this approach in studying the roles of FH in macrophages (FIG. 2c, e, FIG. 9b-Extended Data FIG. 5b). Principal component analysis (PCA) showed a significant divergence of FHIN1 treatment to the other conditions (FIG. 2d).

Tamoxifen-inducible knockout of Fh1 in macrophages (FIG. 9c,d-Extended Data FIG. 5c, d) induced similar bioenergetic changes to FHIN1, demonstrated by reduced ATP/AMP and P-creatine/creatine ratios, although the ATP/ADP ratio was unchanged (FIG. 9e-Extended Data FIG. 5e). TCA cycle rewiring was also observed in Fh1^{-/-} macrophages, although to a lesser extent than with FHIN1 (FIG. 9f-Extended Data FIG. 5f). Compensatory remodelling during initial genetic inactivation of FH may buffer some of the acute changes observed with FHIN1²². Importantly however, fumarate and 2SC levels were increased in Fh1^{-/-} macrophages (FIG. 2f, FIG. 9g-Extended Data FIG. 5g), supporting our parallel use of FHIN1 and Fh1^{-/-} macrophages.

Confirming previous reports⁹, DMF, and to a lesser extent FHIN1, suppressed glycolysis (FIG. 9h-Extended Data FIG. 5h). GAPDH is reportedly inhibited by fumarate-mediated succination^{9,23}. Consistently, FHIN1 increased the glyceraldehyde 3-phosphate (G3P)/2/3-phosphoglycerate (2/3-PG) ratio (FIG. 9i-Extended Data FIG. 5i), suggesting that endogenous fumarate accumulation may impair GAPDH activity. This provides further evidence that FH impairment leads to modulation of cytosolic processes.

As FHIN1 impaired respiration, we examined further mitochondrial parameters. We first observed increased reactive oxygen species (ROS) production in cells treated with FHIN1 but not DMF (FIG. 2g). FHIN1 treatment also increased staining intensity of the mitochondrial membrane potential (MMP)-dependent dye mitotracker RED (mtRED) (FIG. 9j, k-Extended Data FIG. 5j, k). Tetramethylrhodamine methyl ester (TMRM) staining confirmed this result, as FHIN1 significantly increased staining while DMF had no effect (FIG. 2h). Similarly, Fh1^{-/-} macrophages had increased MMP, as previously reported in kidney epithelial cells²⁴ (FIG. 2h). We also observed a decreased aconitate/citrate ratio in FHIN1-treated macrophages, indicative of impairment in the fumarate-and redox-sensitive TCA cycle enzyme aconitase²⁵ (FIG. 2i). Although the GSSG/GSH ratio was unchanged, FHIN1 led to a depletion of total glutathione (FIG. 2j), consistent with fumarate-mediated glutathione depletion^{26,27}. These data suggest that FH inhibition induces profound redox stress responses.

### FH Maintains Appropriate Cytokine Responses

To determine whether FH regulates macrophage activation and effector responses, we performed RNA sequencing and proteomics to assess changes in the transcriptome and proteome of FHIN1-treated BMDMs. Geneset enrichment analysis (GSEA) identified an expected suppression in genes associated with metabolism, but FHIN1 also decreased expression of inflammatory pathways, including IL-1 and IL-10 signalling (FIG. 3a). Increased expression of the heme-regulated inhibitor (HRI) stress response, amino acid metabolism and tRNA aminoacylation was also observed (FIG. 3a), consistent with previous reports¹⁶. Further overrepresentation analysis (ORA) of RNAseq data revealed TNF-α signalling to be the most highly upregulated pathway in our analysis (FIG. 3b).

Comparing FHIN1 with DMF on cytokine readouts allowed us to determine the role of protein succination following FH inhibition. Validating our transcriptomic analysis, FHIN1 and DMF decreased IL-10 release and expression, while TNF-α release and expression were increased (FIG. 3c, FIG. 10a-Extended Data FIG. 6a). Both compounds also reduced IL-1β expression and IL-6 release (FIG. 10b-Extended Data FIG. 6b), consistent with previous reports^{10,28}, demonstrating widespread regulation of cytokine expression.

The less electrophilic fumarate ester, monomethyl fumarate (MMF), exhibited the same effects on II10 and Tnfa expression (FIG. 3d), supporting a role for their regulation by fumarate. Shared transcriptomic changes of FHIN1 and DMF demonstrated strong downregulation of the ERK1/2 cascade and PI3K signalling (FIG. 3e). A similar transcriptional fingerprint has been observed in FH-deficient leiomyomas²⁹. We also observed increased amino acid metabolism and transport, and autophagy transcripts (FIG. 10c-Extended Data FIG. 6c). Upon LPS stimulation, IL-10 is regulated by ERK1/2 and PI3K-induced AP-1 activation³⁰, suggesting that downregulation of this signalling axis by FHIN1 and DMF may repress IL-10. However, we did not observe changes in the upstream kinases (AKT, JNK, ERK and p38) which converge on AP-1 activation, (FIG. 10d-Extended Data FIG. 6d). Although we did observe reduced Jun expression in our transcriptomics dataset (FIG. 10e-Extended Data FIG. 6e), this could indicate reduced autoregulation by AP-1³¹. In this dataset, Fos was not reduced (FIG. 10f-Extended Data FIG. 6f).

Interestingly, the thiol precursor N-acetyl cysteine (NAC) abrogated the suppression of II10 by FHIN1 and DMF (FIG. 3f). The free thiols of NAC and its products would react with and sequester fumarate, thereby reducing the modification of protein thiols and suggesting that suppression of IL-10 results from a redox-dependent succination event. The electrophile sulforaphane has been shown to reduce AP-1 activation via modification of Cys-154 on c-Fos³². We therefore investigated if FHIN1 or DMF may affect c-Fos activation, despite upstream regulators remaining unaffected. Using a c-Fos transcription factor assay, we found that FHIN1 and DMF strongly impaired c-Fos activation (FIG. 3g), providing evidence of direct regulation of c-Fos, potentially through S-alkylation.

IL-10 signalling has been shown to repress TNF-α expression³³. We confirmed this using an IL-10 receptor (CD210) blocking antibody targeting IL-10-mediated STAT3 phosphorylation, leading to augmented LPS-induced TNF-α release (FIG. 3h, FIG. 10g-Extended Data FIG. 6g). We then examined whether recombinant IL-10 supplementation could rescue the increase in TNF-α. Indeed, with IL-10, FHIN1 failed to impair STAT3 phosphorylation or augment TNF-α production (FIG. 3i, j), indicating that the FHIN1- and DMF-driven induction of TNF-α is dependent on the suppression of IL-10.

Confirming the role of FH in regulating this axis, inducible deletion of Fh1 in macrophages from heterozygous Fh1^{+/-} or homozygous Fh1^{-/-} mice (Extended Data FIG. 5c, d, FIG. 10h-Extended Data FIG. 6h) resulted in decreased IL-10 expression and release (FIG. 3k) and increased TNF-α release (FIG. 3I). Furthermore, FHIN1 also suppressed IL10 expression and increased TNFA expression in LPS-stimulated human peripheral blood mononuclear cells (PBMCs) (FIG. 3m) and macrophages (FIG. 3n), indicating that the FH-regulated IL-10/TNF-α axis is also active in human cells. Establishing the role of LPS-driven fumarate accumulation on release of these cytokines, AOAA, which reduces fumarate accumulation (FIG. 1h), modestly increased and reduced IL-10 and TNF-α release respectively (FIG. 10i-Extended Data FIG. 6i), indicating that an increase in ASS1, which results in fumarate accumulation, mildly regulates IL-10 and TNF-α production. These effects are accentuated by pharmacological or genetic inhibition of FH, leading to increased fumarate accumulation (FIG. 10j-Extended Data FIG. 6j). Therefore, sustained expression and activity of FH may be viewed as protective against excessive fumarate accumulation and dysregulated production of IL-10 and TNF-α.

FH inhibition also resulted in the activation of an NRF2 and ATF4 stress response in macrophages (FIG. 11a-Extended Data FIG. 7a), in line with previous observations in epithelial cells¹⁶. Proteomic analysis revealed that the inflammation-associated hormone GDF15³⁴⁻³⁶ is one of the most significantly increased proteins with FHIN1 and DMF, while FHIN1 also increased the recently identified mitochondrial glutathione importer, SLC25A39³⁷, reinforcing the mitochondrial redox perturbation (FIG. 11b, c-Extended Data FIG. 7b, c). Validating our proteomics data, FH inhibition drove GDF15 release from macrophages (FIG. 11d-Extended Data FIG. 7d). Both ATF4 and NRF2 have been reported to regulate GDF15 in different contexts^{35,38}, and silencing of each revealed that FHIN1-driven GDF15 release was partly NRF2-but not ATF4-dependent (FIG. 11e, f-Extended Data FIG. 7e, f). This work defines two previously unappreciated signalling axes linked to FH inhibition, uncovering its role in the regulation of IL-10/TNF-α and GDF15. The recent developments identifying GDF15 as a mediator of immune tolerance, and the anti-inflammatory properties of colchicine and NSAIDS^{38,39}, suggest that protective effects of DMF in models of inflammation could be via GDF15. Additionally, increased TNF-α levels potentially explain adverse events reported with fumarate esters⁴⁰. Mechanistically, suppression of IL-10 may also explain why fumarate esters promote enhanced TNF-α production during trained immunity, in addition to reported epigenetic changes⁴¹.

### FH Restrains mtRNA-Driven IFN-β Release

RNAseq analysis of type I interferon (IFN) response genes revealed divergent effects on IFN expression and signalling with FH inhibition, including an upregulation in Ifnb1 (IFN-β) expression and several interferon-stimulated genes (ISGs), such as Irf1, Ifih1, Rsad2 and Ifit2 (FIG. 4a). However, other ISGs, such as Lcn2, were suppressed by FHIN1 and DMF treatment (FIG. 4a & FIG. 12a-Extended Data FIG. 8a). Examination of specific type I IFN signalling components downstream of the interferon-α/β receptor (IFNAR) revealed that both FHIN1 and DMF treatment limited IFN-β-induced signal transducer and activator of transcription 1 (STAT1) and Janus kinase 1 (JAK1) phosphorylation (FIG. 12b-Extended Data FIG. 8b), indicating modest suppression of JAK/STAT signalling. Activation of NRF2 by fumarate and derivatives (FIG. 11-Extended Data FIG. 7) may be responsible⁴². Indeed, Ifnb1 expression was increased with FHIN1 and DMF following Nrf2 silencing (FIG. 12c, d-Extended Data FIG. 8c, d), suggesting that Nrf2 restrains interferon transcription.

Strikingly, FHIN1, but not DMF or MMF, was found to increase IFN-β release from LPS-stimulated macrophages (FIG. 4b, c). This was independent of NAC-sensitive redox stress (FIG. 12e-Extended Data FIG. 8e), and was not due to augmented TLR4 signalling, as LPS-induced TRAF3 levels and IL-1β expression were not increased by FHIN1 (FIG. 12f, g-Extended Data FIG. 8f, g). FHIN1 and DMF did modestly augment LPS-induced p65 phosphorylation (FIG. 12h-Extended Data FIG. 8h), which may contribute to increased TNF-α release⁴³. Given FH inhibition causes mitochondrial stress (FIG. 2) which is associated with the release of immunostimulatory mitochondrial nucleic acids⁴⁴⁻⁴⁶, we hypothesised that the IFN response was driven by cytosolic nucleic acid sensors, such as cGAS. To support this, FH deficient-hereditary leiomyomatosis and renal cell cancer (HLRCC) tumours exhibit changes in mitochondrial DNA (mtDNA)²². We first used ethidium bromide (EtBr) to deplete mtDNA⁴⁷ (FIG. 12i-Extended Data FIG. 8i) before treating cells with FHIN1 and LPS. We found that FHIN1 no longer boosted LPS-induced IFN-β release in the presence of EtBr (FIG. 4d), indicating that increased IFN-β release with FHIN1 may be mtDNA-dependent. We subsequently found that FHIN1 caused an increase in both mtDNA and mtRNA in cytosolic extracts (FIG. 4e, FIG. 12j-Extended Data FIG. 8j). Given the established role of mtDNA in driving IFN responses^{44,45}, we examined whether the cGAS-STING or TLR9 DNA-sensing pathways were required for the increase in IFN-β. However, neither use of the STING inhibitor C-178⁴⁸ nor silencing of Cgas (cGAS) or Tmem173 (STING) had any effect on FHIN1-driven IFN-β induction (FIG. 12k-n-Extended Data FIG. 8k-n). Targeting TLR9 using the competitive inhibitor ODN 2088⁴⁹ or using siRNA also had no effect on this response (FIG. 12k-n-Extended Data FIG. 8k-n). Suppression of Tmem173 expression by FHIN1 and DMF (FIG. 12o-Extended Data FIG. 8o) may explain why cGAS-STING signalling is redundant in our model, even in the presence of cytosolic mtDNA. ETC inhibition, as we observe with FHIN1 treatment, has also been shown to inhibit STING activation⁵⁰.

Since cytosolic mtRNA was also increased by FHIN1 (FIG. 4e), we performed immunofluorescence staining with an antibody specific for double-stranded RNA (dsRNA). Mitochondrial RNA has previously been shown to drive an IFN response in human cells^{51,52}, and is known to be particularly immunostimulatory⁵³. FHIN1 treatment led to an accumulation of dsRNA relative to DMSO control (FIG. 4f). We subsequently co-treated cells with FHIN1 and IMT1, the mitochondrial RNA polymerase (POLRMT) inhibitor. The increase in mtRNA with FHIN1 was observed in the cytosolic fraction but not in the whole cell fraction and was inhibited in both by co-treatment with IMT1 (FIG. 12p,q-Extended Data FIG. 8p, q). Importantly, IMT1 also abrogated the FHIN1-mediated boost in IFN-β release (FIG. 12r-Extended Data FIG. 8r), implicating the role of mtRNA in driving this response. Mitochondrial ssRNA, resulting from a decline in mitochondrial integrity, has also been implicated in driving TLR7-dependent IFN signalling^{54,55}. We subsequently silenced Tlr7 or the dsRNA sensors Ddx58 (RIG-I) and Ifih1 (MDA5) (FIG. 13a,b-Extended Data FIG. 9a, b), all of which abrogated the boost in IFN-β release observed with FH inhibition (FIG. 4g, h), confirming a non-redundant requirement of these sensors and mtRNA, rather than mtDNA, for the FHIN1-driven IFN response. Knockdown of the cell surface dsRNA sensor Tlr3 did not affect the augmentation in IFN-β release (FIG. 13c-Extended Data FIG. 9c). RIG-I and MDA5, although predominantly described as dsRNA sensors, can also bind ssRNA⁵⁶, indicating that the IFN response following FH inhibition is likely driven by a mixture of dsRNA and ssRNA species. It is notable that FHIN1 also reduced Ddx58 but not Ifih1 expression, which may warrant further investigation (FIG. 13b-Extended Data FIG. 9b). The signalling events downstream of RIG-I/MDA5 activation include mitochondrial antiviral signalling protein (MAVS) oligomerisation, followed by recruitment and phosphorylation of TANK-binding kinase 1 (TBK1). We observed MAVS oligomerisation and increased TBK1 phosphorylation with FHIN1 treatment (FIG. 4i, FIG. 13d-Extended Data FIG. 9d). Intriguingly, MAVS knockout did not impair the induction of IFN-β by FHIN1 (FIG. 13e-Extended Data FIG. 9e), perhaps indicating that compensatory TLR7 signalling is sufficient to drive type I IFN following FH inhibition with chronic MAVS deficiency.

Previously, we demonstrated that FH inhibition causes mitochondrial stress (FIG. 2). Changes in MMP have previously been correlated with increased type I IFN release⁵⁷, thus we hypothesised that disturbances in MMP may be linked to mtRNA release and IFN-β induction following FH inhibition. To support this, we induced changes in MMP by using the ATP synthase inhibitor oligomycin A, which boosted MMP, the K⁺ ionophore valinomycin A, which non-significantly reduced MMP, or the uncoupler CCCP, which significantly dissipated MMP (FIG. 13f, h-Extended Data FIG. 9f, h). All treatments boosted LPS-driven IFN-β release, akin to FHIN1 (FIG. 13g, h-Extended Data FIG. 9g, h). MMF, which does not increase LPS-induced IFN-β expression (FIG. 4c), did not affect MMP (FIG. 13i-Extended Data FIG. 9i). Oligomycin treatment led to an accumulation of dsRNA to a similar extent to that observed in cells treated with FHIN1 or transfected with dsRNA (poly (I:C)), and increased mtRNA release into the cytosol (FIG. 13j-l-Extended Data FIG. 9j-l). Valinomycin treatment similarly drove dsRNA accumulation (FIG. 13m. n-Extended Data FIG. 9m, n), indicating that MMP-altering compounds induce an accumulation of mtRNA. As we also observed an increase in cytosolic mtDNA levels following oligomycin treatment (FIG. 13l-Extended Data FIG. 9l), it is still possible that IFN responses following oligomycin/valinomycin/CCCP treatment are not exclusively driven by mtRNA. mtRNA release from chondrocytes has recently been implicated in activating the immune response and promoting osteoarthritis⁵⁸. As such, mitochondrial damage and nucleic acid release are emerging as key pathogenic processes that may underlie many immune-mediated diseases.

Tamoxifen-inducible Fh1^{-/-} BMDMs released more IFN-β upon LPS stimulation than their Fh1^{+/+} counterparts (FIG. 4j). We also detected increased dsRNA accumulation in Fh1^{-/-} BMDMs (FIG. 4k, FIG. 13o-Extended Data FIG. 9o) which, coupled with the fact that deletion of Fh1 also drives mitochondrial membrane hyperpolarisation (FIG. 2h), demonstrate that both genetic and pharmacological targeting of FH drive similar mitochondrial retrograde type I IFN stress responses.

We next considered whether this response could be applied to an endogenous model of LPS activation in the absence of pharmacological or genetic inactivation of FH. Given LPS-induced FH suppression occurs predominantly during late-phase LPS stimulation (24-48 h) (FIG. 1g), FH suppression at this time point may drive membrane hyperpolarisation and the release of mtRNA. MMP was significantly increased following 48 h LPS stimulation, but not following 4 h or 24 h stimulation (FIG. 14a-Extended Data FIG. 10a). Although dsRNA did not accumulate following acute (4 h) LPS stimulation (FIG. 14j, k-Extended Data FIG. 9j, k), we did observe increased dsRNA staining following 24 h and 48 h LPS stimulation (FIG. 14b, c-Extended Data FIG. 10b, c). Ddx58 and Ifih1 expression is LPS-inducible (FIG. 14d-Extended Data FIG. 10d), which may suggest that RIG-I/MDA5 signalling is required during LPS stimulation. Indeed, silencing of Ddx58 and Ifih1 reduced both 24 h and 48 h LPS-induced Ifnb1 expression (FIG. 4l), indicating that Ifnb1 transcription during late-phase LPS stimulation is maintained by mtRNA release. These results demonstrate that the mitochondrial retrograde type I IFN response, which we initially unmasked by pharmacologically or genetically targeting FH during early LPS signalling, is active endogenously during late-phase LPS activation with potential implications for chronic inflammation, for example during ageing⁵⁹.

To determine whether FH inhibition leads to similar effects in vivo, we injected mice with FHIN1 or DMF prior to administration of LPS, and measured IFN-β release into the serum. FHIN1 increased LPS-induced IFN-β release, while DMF had no effect (FIG. 4m), indicating that FH inhibition leads to a similar IFN response in vivo which may have effects on bystander cells. We also treated human PBMCs with FHIN1 or DMF prior to LPS stimulation and observed similar effects, as FHIN1 boosted, while DMF suppressed LPS-induced IFN-β release (FIG. 4n).

We hereby describe a mitochondrial retrograde signalling pathway leading from FH inhibition to mitochondrial membrane hyperpolarisation and mtRNA release (Supplementary FIG. 1). Mitochondrial stress may be an underlying mechanism that contributes to type I IFN release in interferonopathies such as systemic lupus erythematosus (SLE). It has previously been demonstrated that PBMCs from SLE patients have impaired mitochondrial function and altered MMP^{60,61}. We therefore examined FH expression in the whole blood of SLE patients and found significant suppression of FH compared to healthy control samples (FIG. 4o). Autoantibodies to dsRNA, as well as dsDNA, have been detected in SLE patients^{62,63}. However, it is unclear whether FH suppression is a cause or consequence of increased IFN signalling, as Fh1 can also be inhibited by IFN-β stimulation in BMDMs (FIG. 14e-Extended Data FIG. 10e). A negative feedback loop may exist whereby suppression of FH leads to type I IFN release, which feeds back to further suppress FH. FH suppression has previously also been linked to multiple sclerosis progression⁶⁴ and, in parallel to our work, has been shown to promote a type I IFN response in kidney epithelial cells and HLRCC tumours (Zecchini, Paupe et al., under revision). This study and ours implicate roles for FH in nucleic acid release, which may contribute to inflammation-driven tumorigenesis and as a potential host defence mechanism in the context of viral infection. Finally, the recent demonstration of aberrant dsRNA editing due to ADAR1 deficiency leading to MDA5 activation as a mechanism of common inflammatory diseases also points to the clinical relevance of endogenously produced dsRNA, suggesting that targeting this pathway may yield novel anti-inflammatory strategies⁶⁵.

The invention will now be described by the way of the following non-limiting examples.

### EXAMPLES

### Materials And Methods

### Animal Details

All mice were on a C57BL/6JOIaHsd background unless stated below. Wild-type (WT) mice were bred in-house. The inducible Fh1^{+/fl} and Fh1^{fl/fl} mice were generated on the C57BL/6 genetic background and their hind legs were generously donated by Dr. Christian Frezza (University of Cambridge, UK). Vehicle (ethanol) treated Fh1^{+/+} and Fh1^{-/l} were used as controls. Upon treatment with 4-hydroxy tamoxifen, Cremediated chromatin excision results in the loss of either one (Fh1^{+/-}) or both (Fh1^{-/-}) copies of Fh1, thus generating either heterozygous or null animals. Hind legs from WT and Mavs^{-/-} mice were generously donated by Dr Cecilia Johansson (Imperial College London, UK). These strains, originally obtained from S. Akira (World Premier International Immunology Frontier Research Center, Osaka University, Osaka, Japan), were Ifna6^{gfp/+} but since Ifna6 expression was not a primary readout the mice are designated as WT and Mavs^{-/-}. In vitro experiments were performed with BMDMs isolated from 6-18-week-old female and male mice. Although we did not use statistical methods to calculate sample size, we decided to use a minimum of 3 biological replicates per experiment to account for biological variability, considering the 3 Rs principle and the fact that most experiments were performed in primary murine macrophages from inbred mice. All in vitro treatment groups were randomly assigned. In vitro and in vivo experiments were not blinded due to lack of available experimenters with required expertise. In vivo models were performed with 6-week-old male mice and littermates were randomly assigned to experimental groups. Animals were maintained under specific pathogen-free conditions in line with Irish and European Union regulations. All animal procedures were ethically approved by the Trinity College Dublin Animal Research Ethics Committee prior to experimentation and conformed with the Directive 2010/63/EU of the European Parliament.

### Generation of Murine BMDMs

6-18-week-old mice were euthanised in a CO₂ chamber, and death was confirmed by cervical dislocation. Bone marrow was subsequently harvested from the tibia, femur and ilium and cells were differentiated in DMEM containing L929 supernatant (20%), foetal calf serum (FCS) (10%), and penicillin/streptomycin (1%) for 6 days, after which cells were counted and plated at 0.5×10⁶ cells/ml unless otherwise stated. BMDMs were plated in 12-well cell culture plates and left overnight to adhere.

### Isolation of Human PBMCs

Human blood samples from healthy donors were collected and processed at the School of Biochemistry and Immunology in TBSI (TCD). Blood samples were obtained anonymously and written informed consent for the use of blood for research purposes has been obtained from the donors. All the procedures involving experiments on human samples have been approved by the School of Biochemistry and Immunology Research Ethics Committee (TCD). Experiments were conducted according to the TCD guide on good research practice, which follows the guidelines detailed in the National Institutes of Health Belmont Report (1978) and the Declaration of Helsinki. 30 ml whole blood was layered on 20 ml Lymphoprep (Axis-Shield), followed by centrifugation for 20 mins at 400×g with the brake off, after which the upper plasma layer was removed and discarded. The layer of mononuclear cells at the plasma-density gradient medium interface was retained, and 20 ml PBS was added. Cells were centrifuged for 8 mins at 300×g and the resulting supernatant was removed and discarded. The remaining pellet of mononuclear cells was resuspended, counted, and plated at 1×10⁶ cells/ml in RPMI supplemented with FCS (10%) and penicillin/streptomycin (1%).

### Generation of Human Macrophages

PBMCs were taken, and CD14+ monocytes were isolated using MagniSort Human CD14 Positive Selection Kit (Thermo Fisher) according to the manufacturer's protocol. CD14 Monocytes were then differentiated in T-175 flasks in RPMI containing FCS (10%), penicillin/streptomycin (1%) and recombinant human M-CSF (1:1000). After 6 days, the supernatant was discarded, cells were scraped and counted, and human monocyte-derived macrophages (hMDMs) were plated in 12-well plates at 1×10⁶ cells/mL RPMI containing FCS (10%) and penicillin/streptomycin (1%).

Whole Blood Isolation from SLE Patients

All SLE patients (as per ACR diagnostic criteria) were recruited from Cedars-Sinai Medical Center, CA, USA. Age-and sex-matched healthy donors who had no history of autoimmune diseases or treatment with immunosuppressive agents were included. All participants provided informed written consent and the study received prior approval from the institutional ethics review board (IRB protocol. 19627). Blood was collected into PAXgene RNA tubes (2.5 mL blood+6.9 mL buffer) and stored at −80° C. Before isolation of RNA, the tubes were thawed at room temperature for 16 h. Total RNA was isolated using the PAXgene Blood RNA Kit according to manufacturer's recommendations (PreAnalytix GmbH, 08/2005, REF: 762174).

### Reagents

LPS from Escherichia coli, serotype EH100 (ALX-581-010-L001), was purchased from Enzo Life Sciences. High molecular weight poly (I C) (tlrl-pic) and 2′-3′-cGAMP (tlrl-nacga23) were purchased from Invivogen. Recombinant mouse IFN-β1 (581302) and recombinant mouse IL-10 (417-ML-005/CF) were purchased from Biolegend. ATP disodium salt (A2383), dimethyl sulfoxide (DMSO) (D8418), aminooxyacetic acid (AOAA) (C13408), valinomycin (V3639), 4-hydroxytamoxifen (H6278) and N-acetyl cysteine (NAC) (A7250) were purchased from Sigma Aldrich. Oligomycin A from Streptomyces diastatochromogenes (M02220) was purchased from Fluorochem. Fumarate hydratase-IN-1 (FHIN1) (HY-100004), dimethyl fumarate (DMF) (HY-17363), monomethyl fumarate (MMF) (HY-103252), IMT1 (HY-134539) and C-178 (HY-123963) were purchased from MedChemExpress. CPG ODN 1826 (130-100-274) and ODN 2088 (130-105-815) were purchased from Miltenyi Biotec. CCCP (M20036) was purchased from Thermo Fisher.

### Compound Treatments

All compounds used DMSO as a vehicle except for 4-hydroxy tamoxifen (EtOH), NAC (PBS), and AOAA for tracing experiments (Media). LPS was used at a concentration of 100 ng/ml for indicated timepoints (2, 3, 4, 6, 8, 24, 48 h). FHIN1 (10 or 20 μM), MMF (50 or 100 μM), DMF (25 μM), AOAA (5 mM), oligomycin (10 μM), CCCP (50 μM) NAC (1 mM), and IMT1 (10 μM) pre-treatments were performed for 3 h prior the addition of LPS. Cells were treated with valinomycin (10 nM) 15 mins before LPS stimulation. Anti-CD210 or IgG control (10 μg/ml) antibodies were added to cells 1 h prior to LPS stimulation. Recombinant mouse IL-10 protein (100 ng/ml) was added to cells at the same time as LPS. Cells were treated with IFN-B1 (220 ng/ml) for 3 h. Cells were treated with C-178 (1 μM) 1 h prior to LPS stimulation or transfection with 2′3′-cGAMP (1.5 μg/ml) for 4 hrs to achieve cGAS-STING activation. Cells were treated with ODN 2088 (1 μM) for 1 hr prior to LPS stimulation or transfection with CPG ODN 1826 (1.5 μg/ml) to achieve TLR9 activation. 3 different timepoints of 4-hydroxy tamoxifen (TAM) (600 nM or 2 μM) or EtOH treatment were performed-these are specified in the individual figure legends. For ‘48 h’ treatments EtOH/TAM was added on day 5 of 6 during the BMDM differentiation protocol. On day 6 they were plated with EtOH/TAM (left overnight) and treated the following day. For ‘72 h’ treatments EtOH/TAM was added on day 4 of 6 during the BMDM differentiation protocol. On day 6 they were plated with EtOH/TAM (left overnight) and treated the following day. For ‘96 h’ treatments EtOH/TAM was added on day 4 of 6 during the BMDM differentiation protocol. On day 6 they were plated with EtOH/TAM and treated 2 days later.

### Antibodies

Working dilutions of antibodies were 1/1000 unless otherwise stated. Anti-mouse Lamin B1 (12586), STAT1 (9172), p-STAT1 (9167), JAK1 (3344), p-JAK1 (3331), TBK1 (3504), p-TBK1 (5483), STAT3 (30835), p-STAT3 (9145), FH (4567), ASS1 (70720), α-tubulin (2144), α-tubulin (3873), MAVS (4983), ATF4 (11815), p-AKT (13038), AKT (2920), p-JNK (9255), JNK (9252), p-ERK1/2 (9101), ERK1/2 (4695), p-p38 (4511), p-38 (9212), TRAF3 (4729), p-p65 (3033) and GAPDH (2118) antibodies were purchased from Cell Signaling. Anti-goat IL-1B (AF-401-NA) was purchased from R&D. Anti-2SC antibody was kindly provided by Dr. Norma Frizzell (University of South Carolina, US). Anti-mouse B-actin antibody ( 1/5000) (A5316) was purchased from Sigma Aldrich. Horseradish peroxidase (HRP)-conjugated anti-mouse (115-035-003), anti-goat (705-035-003) and anti-rabbit (111-035-003) immunoglobulin G (IgG) antibodies (all 1/2000) were purchased from Jackson Immunoresearch. Anti-mouse CD210 (112710) and anti-mouse IgG (406601) antibodies (both 10 μg/ml) were purchased from Biolegend. Anti-dsRNA antibody (clone rJ2, 1/60) was purchased from Merck (MABE-1134). Alexa Fluor 488 goat anti-mouse IgG1 antibody (A21121) was purchased from Invitrogen. Details of antibody validation are given in Table S1.

### RT-qPCR

RNA extraction from cells was carried out using a Purelink™ RNA kit (Invitrogen) according to the manufacturer's instructions. BMDMs were treated as required, and following treatment were instantly lysed in 350 μl RNA lysis buffer. Isolated RNA was quantified using a NanoDrop 2000 spectrophotometer, and RNA concentration was normalised to the lowest concentration across all samples with RNAse-free water. If necessary, samples were DNAse-treated after quantification using DNAse I (Thermo Fisher) according to the manufacturer's instructions. Isolated RNA samples were normalised and converted into cDNA using the High-Capacity cDNA Reverse Transcription Kit (Thermo Fisher) according to manufacturer's instructions. 10 μl of RNA (at a maximum concentration of 100 ng/μl) was added to 10 μl of reverse transcription master mix to complete the reaction mixture. Real-time quantitative PCR was performed on the cDNA generated in the previous step, using primers designed in-house and ordered from Eurofins Genomics, as detailed in Table S2. The reaction was performed in a 96-well qPCR plate by a 7500 Fast Real-Time PCR machine (Thermo Fisher). Relative expression (2^{-ΔΔCT}) was calculated from the C_{T} values for each sample and gene of interest.

### RNA Interference (RNAi)

Pre-designed silencer select siRNAs for Cgas (s103166), Tmem173 (s91058), Tlr3 (s100579), Tlr9 (s96268), As/(s99640), Tlr7 (s100720), Ddx58 (s106376), Ifih1 (s89787), Nrf2 (s70522), Atf4 (s62689) and negative control (4390843) were ordered from Thermo Fisher. siRNA sequences are given in Table S2. Cells were transfected with 50 nM siRNA using 5 μl lipofectamine RNAiMAX according to manufacturer's instructions (Thermo Fisher). Cells were transfected in medium without serum and antibiotics which was replaced with complete medium 8 hours later. Cells were subsequently left for at least a further 12 hours prior to treatment.

### Immunofluorescence

Cells were plated on 20 mm cover slips in 12-well plates. Cells were treated as required and Mitotracker Red CMXRos (100 nM, Thermo Fisher), was added to medium 30 mins prior to end of cell treatments. After 30 min incubation, cells were washed three times with warm PBS. Cells were subsequently fixed for 10 mins with 4% paraformaldehyde/PBS at 37° C. Cells were washed three times with PBS and permeabilized for 1 hour in block solution (1% BSA, 22.52 mg/ml glycine, 0.1% tween 20 in PBS). Anti-dsRNA antibody (Merck) was diluted 1/60 in block solution and incubated with cells overnight at room temperature. Cells were washed three times with PBS for 5 mins/wash. A mix containing AF488-conjugated goat anti-mouse IgG1 antibody ( 1/1000) and DAPI ( 1/1000, Thermo Fisher) was subsequently added to cells for 90 mins at room temperature in the dark. Cells were subsequently washed three times with PBS for 5 mins/wash. Cover slips were mounted onto microscope slides using 10-20 μl ProLong Gold antifade reagent (Thermo Fisher). Slides were imaged using a Leica SP8 scanning confocal microscope using 20.0×objective. Images were analysed using the LAS X Life Science Microscope Software Platform (Leica). The same microscope instrument settings were used for all samples and all images were analysed using the same settings. Scale bars=20 μm. Quantification of dsRNA or Mitotracker Red CMXRos signal intensity was performed using the measure function in ImageJ 1.53t (NIH). Mean signal intensity was calculated for individual cells in single colour images and displayed relative to signal intensity of control cells.

### Flow Cytometry

Cells were plated in 12-well plates and treated as desired. CellROX Green (5 μM, Thermo Fisher) or TMRM (20 nM, Thermo Fisher) was added to cells 30 mins prior to end of cell treatments. Cells were washed once in PBS and scraped into 200 μl FACS buffer (2 mM EDTA, 0.5% FCS in PBS). Acquisition of samples was performed on a BD Accuri C6 flow cytometer. The gating strategy used for all flow cytometry experiments consisted of debris exclusion by FSC-A vs SSC-A analysis and subsequent doublet exclusion by FSC-A vs FSC-H analysis. A sample gating strategy is provided in Supplementary FIG. 2. 10,000 cells was acquired per condition. Mean fluorescence intensity (MFI) was calculated for all cells in each condition using FlowJo v10.

### Liquid-Chromatography-Mass Spectrometry (LC-MS)

### Steady-State Metabolomics

BMDMs (3 independent mice) were plated at 0.5×10⁶ cells/well in 12-well plates in technical triplicate per condition, treated as indicated, snap frozen and stored at −80° C. For metabolomics on cytosolic fraction, BMDMs were plated at 10×10⁶ cells/10 cm dish and rapid fractionation was performed as previously reported¹⁶. Metabolite extraction solution (MES) (methanol/acetonitrile/water, 50:30:20 v/v/v) was added (0.5 mL per 1×10⁶ cells) and samples were incubated for 15 min on dry ice. The resulting suspension was transferred to ice-cold microcentrifuge tubes. Samples were agitated for 20 min at 4° C. in a thermomixer and then incubated at −20° C. for 1 h. Samples were centrifuged at maximum speed for 10 min at 4° C. The supernatant was transferred into a new tube and centrifuged again at maximum speed for 10 min at 4° C. The supernatant was transferred to autosampler vials and stored at −80° C. prior to analysis by LC-MS.

HILIC chromatographic separation of metabolites was achieved using a Millipore Sequant ZIC-PHILIC analytical column (5 μm, 2.1×150 mm) equipped with a 2.1×20 mm guard column (both 5 mm particle size) with a binary solvent system. Solvent A was 20 mM ammonium carbonate, 0.05% ammonium hydroxide; Solvent B was acetonitrile. The column oven and autosampler tray were held at 40° C. and 4° C., respectively. The chromatographic gradient was run at a flow rate of 0.200 mL/min as follows: 0-2 min: 80% B; 2-17 min: linear gradient from 80% B to 20% B; 17-17.1 min: linear gradient from 20% B to 80% B; 17.1-22.5 min: hold at 80% B. Samples were randomized and analysed with LC-MS in a blinded manner and the injection volume was 5 μl. Pooled samples were generated from an equal mixture of all individual samples and analysed interspersed at regular intervals within sample sequence as a quality control. Metabolites were measured with a Thermo Scientific Q Exactive Hybrid Quadrupole-Orbitrap Mass spectrometer (HRMS) coupled to a Dionex Ultimate 3000 UHPLC or with Vanquish Horizon UHPLC coupled to an Orbitrap Exploris 240 mass spectrometer (both Thermo Fisher Scientific) via a heated electrospray ionization source.

For Thermo Scientific Q Exactive Hybrid Quadrupole-Orbitrap Mass spectrometer (HRMS) coupled to a Dionex Ultimate 3000 UHPLC, the mass spectrometer was operated in full-scan, polarity-switching mode, with the spray voltage set to +4.5 kV/−3.5 kV, the heated capillary held at 280° C. and the heated electrospray ionization probe held at 320° C. The sheath gas flow was set to 40 units, the auxiliary gas flow was set to 15 units, and the sweep gas flow was set to 0 unit. HRMS data acquisition was performed in a range of m/z=70-900, with the resolution set at 70,000, the AGC target at 1×10⁶, and the maximum injection time (Max IT) at 120 ms. Metabolite identities were confirmed using two parameters: (1) precursor ion m/z was matched within 5 ppm of theoretical mass predicted by the chemical formula; (2) the retention time of metabolites was within 5% of the retention time of a purified standard run with the same chromatographic method. Chromatogram review and peak area integration were performed using the Thermo Fisher software XCalibur Qual Browser, XCalibur Quan Browser software and Tracefinder 5.0 and the peak area for each detected metabolite was normalized against the total ion count (TIC) of that sample to correct any variations introduced from sample handling through instrument analysis. Absolute quantification of 2SC was performed by interpolation of the corresponding standard curve obtained from serial dilutions of commercially available standards (Sigma Aldrich) running with the same batch of samples.

For the Orbitrap Exploris 240 mass spectrometer, MS1 scans, mass range was set to m/z=70-900, AGC target set to standard and maximum injection time (IT) set to auto. Data acquisition for experimental samples used full scan mode with polarity switching at an Orbitrap resolution of 120000. Data acquisition for untargeted metabolite identification was performed using the AcquireX Deep Scan workflow, an iterative data-dependent acquisition (DDA) strategy using multiple injections of the pooled sample. In brief, sample was first injected in full scan-only mode in single polarity to create an automated inclusion list. MS2 acquisition was then carried out in triplicate, where ions on the inclusion list were prioritized for fragmentation in each run, after which both the exclusion and inclusion lists were updated in a manner where fragmented ions from the inclusion list were moved to exclusion list for the next run. DDA full scan-ddMS2 method for AcquireX workflow used the following parameters: full scan resolution was set to 60000, fragmentation resolution to 30000, fragmentation intensity threshold to 5.0e3. Dynamic exclusion was enabled after 1 time and exclusion duration was 10 s. Mass tolerance was set to 5 ppm. Isolation window was set to 1.2 m/z. Normalized HCD collision energies were set to stepped mode with values at 30, 50, 150. Fragmentation scan range was set to auto, AGC target at standard and max IT at auto. Xcalibur AcquireX method modification was on. Mild trapping was enabled.

Metabolite identification was performed in the Compound Discoverer software (v 3.2, Thermo Fisher Scientific). Metabolites were annotated at the MS2 level using both an in-house mzVault spectral database curated from 1051 authentic compound standards and the online spectral library mzCloud. The precursor mass tolerance was set to 5 ppm and fragment mass tolerance set to 10 ppm. Only metabolites with mzVault or mzCloud best match score above 50% and 75%, respectively, and RT tolerance within 0.5 min to that of a purified standard run with the same chromatographic method were exported to generate a list including compound names, molecular formula and RT. The curated list was then used for further processing in the Tracefinder software (v 5.0, Thermo Fisher Scientific), where extracted ion chromatographs for all compounds were examined and manually integrated if necessary. False positive, noise or chromatographically unresolved compounds were removed. The peak area for each detected metabolite was then normalized against the total ion count (TIC) of that sample to correct any variations introduced from sample handling through instrument analysis. The normalized areas were used as variables for further statistical data analysis. Statistical analysis was performed using MetaboAnalyst 5.0⁶⁶.

### Stable Isotope-Assisted Tracing

BMDMs (3 independent mice) were plated at 0.5×10⁶ cells/well in 12-well plates in technical triplicate per condition, treated as indicated in glutamine-free DMEM supplemented with U-¹³C-glutamine or ¹⁵N₂-glutamine, respectively. For ¹³C- and ¹⁵N-tracing analysis, the theoretical masses of ¹³C and ¹⁵N isotopes were calculated and added to a library of predicted isotopes in Tracefinder 5.0. These masses were then searched with a 5-ppm tolerance and integrated only if the peak apex showed less than 1% deviation in retention time from the [U-¹²C or ¹⁴N] monoisotopic mass in the same chromatogram. The raw data obtained for each isotopologue were corrected for natural isotope abundances the using AccuCor algorithm (https://github.com/Iparsons/accucor) before further statistical analysis.

### EtBr Treatment

BMDMs were plated in the presence or absence of ultrapure ethidium bromide (100 ng/ml) and incubated for a further 6 days prior to treatment. Depletion of mtDNA was determined by genomic DNA isolation followed by qPCR using primers specific for areas of mitochondrial DNA (D-loop) and areas of mtDNA that are not inserted into nuclear DNA (Non-NUMT).

c-Fos Activity Assay

BMDMs from 3 mice were plated in 10 cm dishes at 0.5×10⁶ cells/mL and left overnight. Cells were pre-treated with FHIN1 or DMF (3 h) prior to LPS stimulation (4 h). Upon harvesting, nuclear extracts were isolated using a Nuclear Extraction Kit (ab113474) purchased from Abcam. Nuclear extracts were quantified via BCA assay and standardised. c-Fos relative activity was then quantified using the AP1 transcription factor assay purchased from Abcam (Ab207196) according to the manufacturers protocol.

### Fumarate Assay

Analysis of fumarate levels were assessed using a fumarate colorimetric assay kit (Sigma MAK060) that uses an enzyme assay, which results in a colorimetric (450 nm) product proportional to the fumarate present, as per manufacturer's instructions.

### Nitrite Measurement

The Griess Reagent System (Promega G2930) was used according to manufacturer's instructions.

### RNA Sequencing

BMDMs (3 independent mice) were treated as indicated and RNA was extracted as previously detailed. mRNA was extracted from total RNA using poly-T-oligo-attached magnetic beads. After fragmentation, the first strand cDNA was synthesized using random hexamer primers, followed by the second strand cDNA synthesis. The library was checked with Qubit and real-time PCR for quantification and bioanalyzer for size distribution detection. Quantified libraries were pooled and sequenced on the NovaSeq 6000 S4 (Illumina). Differential expression analysis of two conditions/groups was performed using counted reads and the DESeq2 R package⁶⁷. Pathway enrichment analyses were performed as indicated in quantification and statistical analysis.

### Proteomic Analysis

### Sample Preparation

BMDMs (from 5 independent mice) were plated onto 10-cm dishes and treated as indicated. At the experimental endpoint, cells were washed with PBS on ice and centrifuged at 1500 rpm for 5 mins at 4° C. and frozen at −80° C. Cell pellets were lysed, reduced and alkylated in 50 μl of 6M Gu-HCl, 200 mM Tris-HCl PH 8.5, 10 mM TCEP, 15 mM Chloroacetamide by probe sonication and heating to 95° C. for 5 min. Protein concentration was measured by a Bradford assay and initially digested with LysC (Wako) with an enzyme to substrate ratio of 1/200 for 4 h at 37° C. Subsequently, the samples were diluted tenfold with water and digested with porcine trypsin (Promega) at 37° C. overnight. Samples were acidified to 1% TFA, cleared by centrifugation (16,000 g at RT) and approximately 20 μg of the sample was desalted using a Stage-tip. Eluted peptides were lyophilized, resuspended in 0.1% TFA/water and the peptide concentration was measured by A280 on a nanodrop instrument (Thermo). The sample was diluted to 2 μg/5 μl for subsequent analysis.

### MS Analysis

The tryptic peptides were analysed on a Fusion Lumos mass spectrometer connected to an Ultimate Ultra3000 chromatography system (both Thermo Scientific) incorporating an autosampler. 2 μg of de-salted peptides were loaded onto a 50 cm emitter packed with 1.9 μm ReproSil-Pur 200 C18-AQ (Dr Maisch, Germany) using a RSLC-nano uHPLC systems connected to a Fusion Lumos mass spectrometer (both Thermo, UK). Peptides were separated by a 140 min linear gradient from 5% to 30% acetonitrile, 0.5% acetic acid. The mass spectrometer was operated in DIA mode, acquiring a MS 350-1650 Da at 120 k resolution followed by MS/MS on 45 windows with 0.5 Da overlap (200-2000 Da) at 30 k with a NCE setting of 27.

### Data Analysis

Raw files were analysed and quantified by searching against the Uniprot Mus Musculus database using DIA-NN 1.8 (https://github.com/vdemichev/DiaNN). Library-free search was selected, and the precursor ion spectra were generated from the FASTA file using the deep learning option. Default settings were used throughout apart from using “Robust LC (high precision)”. In brief, Carbamidomethylation was specified as fixed modification while acetylation of protein N-termini was specified as variable. Peptide length was set to minimum 7 amino acids, precursor FDR was set to 1%. Subsequently, missing values were replaced by a normal distribution (1.8π shifted with a distribution of 0.3π) to allow the following statistical analysis. Protein-wise linear models combined with empirical Bayes statistics are used for the differential expression analyses. We use the Bioconductor package limma to carry out the analysis using the information provided in the experimental design table.

### Digitonin Fractionation

BMDMs were plated at 0.5×10⁶ cells/well and treated as desired. After treatment, cells were washed once with room temperature PBS, before being scraped on ice into ice cold PBS and pelleted at 500×g for 5 mins at 4° C. Supernatant was removed and discarded, and the pellet was resuspended in 400 μl extraction buffer (150 mM NaCl, 50 mM HEPES pH 7.4, 25 μg/ml digitonin). Samples were then placed in a rotating mixer at 4° C. for 10 mins before centrifugation at 2000×g at 4° C. for 5 mins. The resulting supernatant constituted the cytosolic fraction, from which RNA and DNA were isolated using an AllPrep DNA/RNA Mini Kit (Qiagen). Alternatively, the cytosolic fraction was concentrated using Strataclean Resin (Agilent) and analysed by western blot. The pellet constituted a fraction containing membrane-bound organelles which was lysed in RNA lysis buffer for RNA isolation or lysed in western blot lysis buffer for analysis by western blot. To determine the presence of mtRNA and mtDNA in the cytosol, qPCR was performed using primers specific for mitochondrial D-loop on cDNA which had been reverse-transcribed from RNA isolated from the cytosolic fraction (mtRNA) and on DNA isolated from the cytosolic fraction (mtDNA). In both cases, values were normalised using a housekeeping control gene (β-actin) amplified in cDNA which had been reverse-transcribed from RNA isolated from the membrane-bound fraction.

### MAVS Oligomerisation

BMDMs were plated at 1×10⁶ cells/well in technical triplicate and treated as desired. After treatment, cells were washed twice with 200 μl cold PBS before being lysed in crosslinking lysis buffer (50 mM HEPES, 0.5% triton X-100, 1X protease inhibitor cocktail). Samples were placed on ice for 15 mins. Lysates were centrifuged for 15 mins at 6000×g at 4° C. and the supernatant was removed and frozen down as the ‘soluble fraction.’ 20 μl of the soluble fraction was mixed with 5 μl of sample lysis buffer (0.125 M Tris pH 6.8, 10% glycerol, 0.02% SDS, 5% DTT) and run on a 10% gel. The insoluble pellet was resuspended in HEPES (50 mM) and washed 3 times by centrifuging at 6000×g at 4° C. and removing the supernatant each time. After the final wash, the pellet was resuspended in 500 μl crosslinking buffer (50 mM HEPES, 150 mM NaCl) and disuccinimidyl suberate (DSS, Thermo Fisher, made up in anhydrous DMSO) was added to the final concentration of 2 mM. Immediately following the addition of DSS, the sample was inverted several times and incubated for 45 mins at 37° C. The sample was then centrifuged for 15 mins at 6000×g at 4° C., before the supernatant was removed and the pellet was resuspended in 30 μl sample lysis buffer. The resuspended ‘insoluble fraction’ was subsequently boiled for 5 mins at 95° C. before being run on a gel.

### Seahorse XF Glycolysis Stress Test

Cells were plated at 100,000 cells/well in 100 μl and were left overnight to adhere. Protocol was carried out according to manufacturer's instructions (Agilent). In brief, cells were treated as required, after which medium was replaced with Seahorse medium containing glutamine (2 mM). Cells were then placed in a CO₂-free incubator for 1 hour. Glycolysis stress test was subsequently performed using a Seahorse XFe96 Analyzer (Agilent) with the following injections:
A—Glucose (10 mM)
B—Oligomycin (1 μM)
C—2-DG (50 mM)
   Analysis was performed using Seahorse Wave Software (Agilent). Data shown are representative experiments containing at least 3 pooled biological replicates.

### Seahorse XF Mito Stress Test

Cells were plated at 100,000 cells/well in 100 μl and were left overnight to adhere.

Protocol was carried out according to manufacturer's instructions (Agilent). In brief, cells were treated as required, after which medium was replaced with Seahorse medium containing glutamine (2 mM), glucose (10 mM) and pyruvate (1 mM). Cells were then placed in a CO₂-free incubator for 1 hour. Mito stress test was subsequently performed using a Seahorse XFe96 Analyzer (Agilent) with the following injections:
A—Oligomycin (1 μM)
B—FCCP (1 μM)
C—Rotenone (500 nM)
   Analysis was performed using Seahorse Wave Software (Agilent). Data shown are representative experiments containing at least 3 pooled biological replicates.

### LPS-Induced Inflammation Model

6-week-old male mice were used, and littermates were randomly assigned to experimental groups. Compounds were resuspended in 10% DMSO followed by 90% cyclodextrin/PBS (20% w/v). Mice were injected intraperitoneally with vehicle, FHIN1 or DMF (both 50 mg/kg) at a volume of 200 μl per injection. 1 hour later, mice were injected intraperitoneally with PBS or LPS from E. coli (2.5 mg/kg, Sigma) at a volume of 100 μl per injection. 2 hours later, mice were euthanised and blood was harvested retro-orbitally. Blood was allowed to clot for 30 mins at room temperature before it was centrifuged at 5000×g for 10 mins at 4° C. The serum was removed and IFN-β concentration was determined by ELISA.

### Western Blotting

Supernatant was removed from cells following stimulation and lysates were harvested in 30-50 μl lysis buffer (0.125 M Tris pH 6.8, 10% glycerol, 0.02% SDS, 5% DTT) Lysates were subsequently heated to 95° C. for 5 mins to denature proteins. SDS-PAGE was used to resolve proteins by molecular weight. Samples were boiled at 95° C. for 5 mins prior to loading into a 5% stacking gel. The percentage resolving gel depended on the molecular weight of the given protein. The Bio-Rad gel running system was used to resolve proteins and the Bio-Rad wet transfer system was used for the electrophoretic transfer of proteins onto PVDF membrane. Following transfer, the membrane was incubated in milk powder (5% in TBST) for 1 hr and subsequently incubated in primary antibody rolling overnight at 4° C. Primary antibodies targeting phospho-proteins were diluted in BSA (5% in TBST) as opposed to milk. The membrane was incubated for 1 hr with secondary antibody (diluted in 5% milk powder) at room temperature. Prior to visualisation, the membrane was immersed in WesternBright ECL Spray (Advansta). Protein visualisation took place on a ChemiDoc MPTM Imaging System (Bio-Rad), and both chemiluminescent and white light images were taken. Images were analysed using Image Lab 6.0.1 (Bio-Rad).

### ELISA

DuoSet ELISA kits for IL-1β, TNFα, IL-6, IL-10, and GDF15 were purchased from R&D Systems and were carried out according to the manufacturer's instructions with appropriately diluted cell supernatants added to each plate in duplicate or triplicate. IFN-β was determined using DuoSet ELISA kit from R&D Systems or Abcam (ab252363). Quantikine ELISA kit for IFN-β (R&D Systems) was used for determination of IFN-β concentration in serum samples and from human cells, and these were also carried out according to the manufacturer's instructions. Absorbance at 450 nm was quantified using a FLUOstar Optima plate reader. Corrected absorbance values were calculated by subtracting the background absorbance, and cytokine concentrations were subsequently obtained by extrapolation from a standard curve plotted on GraphPad Prism 9.2.0.

### Quantification and Statistical Analyses

Details of all statistical analyses performed can be found in the figure legends. Data were expressed as mean±standard error of the mean (SEM) unless stated otherwise. Representative western blots are shown. For metabolomics data, MetaboAnalyst 5.0⁶⁶ was used to analyse, perform statistics, and visualise the results. Autoscaling of features (metabolites) was used for heatmap generation. One-way ANOVA corrected for multiple comparisons by the Tukey statistical test was used and a p.adjusted<0.05 was set as the cut-off. For proteomics data, protein signal intensity was converted to a log₂ scale and biological replicates were grouped by experimental condition. Protein-wise linear models combined with empirical Bayes statistics were used for the differential expression analyses. The Bioconductor package limma was used to carry out the analysis using an R based online tool⁶⁸. Data were visualised using a heatmap with autoscaled features (genes) and a Volcano plot, which shows the log₂ fold change on the x axis and the −log₁₀ adjusted p value on the y axis. The proteomics cut-offs for analysis were a log₂FC of 0.5 and a false discovery rate (FDR)<0.05, determined using t statistics. RNA seq cut-offs were set to log₂FC of 1 and an FDR<0.05. Overrepresentation analysis (ORA) of significant changes were assessed using Enrichr⁴ and the Bioconductor package clusterProfiler 4.0 in R (version 3.6.1). Further information on this visualisation method is available⁶⁹. Emapplots were generated using enrichplot package in R (version 3.6.1). GSEA analysis of RNAseq was performed using the Broad Institutes GSEA 4.1.070. Graphpad Prism 9.2.0 was used to calculate statistics in bar plots using appropriate statistical tests depending on the data including one-way ANOVA, two-tailed unpaired t test and multiple t tests. Adjusted p values were assessed using appropriate correction methods, such as Tukey, Kruskal-Wallis, and Holm-Sidak tests. Sample sizes were determined based on previous experiments using similar methodologies. All depicted data points are biological replicates taken from distinct samples, unless stated otherwise. Each figure consists of a minimum of 3 independent experiments from multiple biological replicates, unless stated otherwise. For in vivo studies, mice were randomly assigned to treatment groups. For metabolomics, proteomics and RNA sequencing analyses, samples were processed in random order and experimenters were blinded to experimental conditions.

### Data Availability

Proteomics data from FIG. 1d were previously deposited to the ProteomeXchange Consortium via the PRIDE partner repository with the dataset identifier PXD029155¹¹. All other proteomics, RNA sequencing data and metabolomics data have been deposited to Dryad (doi:10.5061/dryad.6wwpzgn28). All original gel images are provided in the source data file.

### REFERENCES

1. Mills, E. L. et al. Succinate Dehydrogenase Supports Metabolic Repurposing of Mitochondria to Drive Inflammatory Macrophages. Cell 167, 457-470 e413, doi:10.1016/j.cell.2016.08.064 (2016).
2. Mills, E. L. et al. Itaconate is an anti-inflammatory metabolite that activates Nrf2 via alkylation of KEAP1. Nature 556, 113-117, doi:10.1038/nature25986 (2018).
3. Tannahill, G. M. et al. Succinate is an inflammatory signal that induces IL-1beta through HIF-1alpha. Nature 496, 238-242, doi:10.1038/nature11986 (2013).
4. Lampropoulou, V. et al. Itaconate Links Inhibition of Succinate Dehydrogenase with Macrophage Metabolic Remodeling and Regulation of Inflammation. Cell Metab 24, 158-166, doi:10.1016/j.cmet.2016.06.004 (2016).
5. Jha, Abhishek K. et al. Network Integration of Parallel Metabolic and Transcriptional Data Reveals Metabolic Modules that Regulate Macrophage Polarization. Immunity 42, 419-430, doi:10.1016/j.immuni.2015.02.005 (2015).
6. Billingham, L. K. et al. Mitochondrial electron transport chain is necessary for NLRP3 inflammasome activation. Nat Immunol 23, 692-704, doi:10.1038/s41590-022-01185-3 (2022).
7. Mills, E. L., Kelly, B. & O'Neill, L. A. J. Mitochondria are the powerhouses of immunity. Nat Immunol 18, 488-498, doi:10.1038/ni.3704 (2017).
8. Adam, J. et al. Renal cyst formation in Fh1-deficient mice is independent of the Hif/Phd pathway: roles for fumarate in KEAP1 succination and Nrf2 signaling. Cancer Cell 20, 524-537, doi:10.1016/j.ccr.2011.09.006 (2011).
9. Kornberg, M. D. et al. Dimethyl fumarate targets GAPDH and aerobic glycolysis to modulate immunity. Science 360, 449-453, doi:10.1126/science.aan4665 (2018).
10. Humphries, F. et al. Succination inactivates gasdermin D and blocks pyroptosis. Science, doi:10.1126/science.abb9818 (2020).
11. Williams, N. C. et al. Signaling metabolite L-2-hydroxyglutarate activates the transcription factor HIF-1alpha in lipopolysaccharide-activated macrophages. J Biol Chem 298, 101501, doi:10.1016/j.jbc.2021.101501 (2021).
12. Cordes, T. et al. Immunoresponsive Gene 1 and Itaconate Inhibit Succinate Dehydrogenase to Modulate Intracellular Succinate Levels. J Biol Chem 291, 14274-14284, doi:10.1074/jbc.M115.685792 (2016).
13. Sass, E., Blachinsky, E., Karniely, S. & Pines, O. Mitochondrial and cytosolic isoforms of yeast fumarase are derivatives of a single translation product and have identical amino termini. J Biol Chem 276, 46111-46117, doi:10.1074/jbc.M106061200 (2001).
14. Adam, J. et al. A role for cytosolic fumarate hydratase in urea cycle metabolism and renal neoplasia. Cell Rep 3, 1440-1448, doi:10.1016/j.celrep.2013.04.006 (2013).
15. Takeuchi, T., Schumacker, P. T. & Kozmin, S. A. Identification of fumarate hydratase inhibitors with nutrient-dependent cytotoxicity. J Am Chem Soc 137, 564-567, doi:10.1021/ja5101257 (2015).
16. Ryan, D. G. et al. Disruption of the TCA cycle reveals an ATF4-dependent integration of redox and amino acid metabolism. Elife 10, doi:10.7554/eLife.72593 (2021).
17. Hayashi, G. et al. Dimethyl fumarate mediates Nrf2-dependent mitochondrial biogenesis in mice and humans. Hum Mol Genet 26, 2864-2873, doi:10.1093/hmg/ddx167 (2017).
18. Sciacovelli, M. et al. Fumarate is an epigenetic modifier that elicits epithelial-to-mesenchymal transition. Nature 537, 544-547, doi:10.1038/nature19353 (2016).
19. Wang, Y. P. et al. Malic enzyme 2 connects the Krebs cycle intermediate fumarate to mitochondrial biogenesis. Cell Metab 33, 1027-1041 e1028, doi:10.1016/j.cmet.2021.03.003 (2021).
20. Lampropoulou, V. et al. Itaconate Links Inhibition of Succinate Dehydrogenase with Macrophage Metabolic Remodeling and Regulation of Inflammation. 24, 158-166, doi:10.1016/j.cmet.2016.06.004 (2016).
21. Liao, S. T. et al. 4-Octyl itaconate inhibits aerobic glycolysis by targeting GAPDH to exert anti-inflammatory effects. Nat Commun 10, 5091, doi:10.1038/s41467-019-13078-5 (2019).
22. Crooks, D. R. et al. Mitochondrial DNA alterations underlie an irreversible shift to aerobic glycolysis in fumarate hydratase-deficient renal cancer. Sci Signal 14, doi:10.1126/scisignal.abc4436 (2021).
23. Blatnik, M., Frizzell, N., Thorpe, S. R. & Baynes, J. W. Inactivation of glyceraldehyde-3-phosphate dehydrogenase by fumarate in diabetes: formation of S-(2-succinyl) cysteine, a novel chemical modification of protein and possible biomarker of mitochondrial stress. Diabetes 57, 41-49, doi:10.2337/db07-0838 (2008).
24. Tyrakis, P. A. et al. Fumarate Hydratase Loss Causes Combined Respiratory Chain Defects. Cell Rep 21, 1036-1047, doi:10.1016/j.celrep.2017.09.092 (2017).
25. Ternette, N. et al. Inhibition of mitochondrial aconitase by succination in fumarate hydratase deficiency. Cell Rep 3, 689-700, doi:10.1016/j.celrep.2013.02.013 (2013).
26. Sullivan, L. B. et al. The proto-oncometabolite fumarate binds glutathione to amplify ROS-dependent signaling. Mol Cell 51, 236-248, doi:10.1016/j.molcel.2013.05.003 (2013).
27. Zheng, L. et al. Fumarate induces redox-dependent senescence by modifying glutathione metabolism. Nat Commun 6, 6001, doi:10.1038/ncomms7001 (2015).
28. Bambouskova, M. et al. Electrophilic properties of itaconate and derivatives regulate the IkappaBzeta-ATF3 inflammatory axis. Nature 556, 501-504, doi:10.1038/s41586-018-0052-z (2018).
29. Raimundo, N., Vanharanta, S., Aaltonen, L. A., Hovatta, I. & Suomalainen, A. Downregulation of SRF-FOS-JUNB pathway in fumarate hydratase deficiency and in uterine leiomyomas. Oncogene 28, 1261-1273, doi:10.1038/onc.2008.472 (2009).
30. Hu, X. et al. IFN-gamma suppresses IL-10 production and synergizes with TLR2 by regulating GSK3 and CREB/AP-1 proteins. Immunity 24, 563-574, doi:10.1016/j.immuni.2006.02.014 (2006).
31. Angel, P., Hattori, K., Smeal, T. & Karin, M. The jun proto-oncogene is positively autoregulated by its product, Jun/AP-L1. Cell 55, 875-885, doi:10.1016/0092-8674 (88) 90143-2 (1988).
32. Dickinson, S. E. et al. Inhibition of activator protein-1 by sulforaphane involves interaction with cysteine in the cFos DNA-binding domain: implications for chemoprevention of UVB-induced skin cancer. Cancer Res 69, 7103-7110, doi:10.1158/0008-5472.CAN-09-0770 (2009).
33. de Waal Malefyt, R., Abrams, J., Bennett, B., Figdor, C. G. & de Vries, J. E. Interleukin 10(IL-10) inhibits cytokine synthesis by human monocytes: an autoregulatory role of IL-10 produced by monocytes. J Exp Med 174, 1209-1220, doi:10.1084/jem.174.5.1209 (1991).
34. Luan, H. H. et al. GDF15 Is an Inflammation-Induced Central Mediator of Tissue Tolerance. Cell 178, 1231-1244 e1211, doi:10.1016/j.cell.2019.07.033 (2019).
35. Day, E. A. et al. Metformin-induced increases in GDF15 are important for suppressing appetite and promoting weight loss. Nat Metab 1, 1202-1208, doi:10.1038/s42255-019-0146-4 (2019).
36. Coll, A. P. et al. GDF15 mediates the effects of metformin on body weight and energy balance. Nature 578, 444-448, doi:10.1038/s41586-019-1911-y (2020).
37. Wang, Y. et al. SLC25A39 is necessary for mitochondrial glutathione import in mammalian cells. Nature 599, 136-140, doi:10.1038/s41586-021-04025-w (2021).
38. Weng, J. H. et al. Colchicine acts selectively in the liver to induce hepatokines that inhibit myeloid cell activation. Nat Metab 3, 513-522, doi:10.1038/s42255-021-00366-y (2021).
39. Eisenstein, A. et al. Activation of the transcription factor NRF2 mediates the anti-inflammatory properties of a subset of over-the-counter and prescription NSAIDs. Immunity 55, 1082-1095 e1085, doi:10.1016/j.immuni.2022.04.015 (2022).
40. Asadullah, K. et al. Influence of monomethylfumarate on monocytic cytokine formation—explanation for adverse and therapeutic effects in psoriasis? Arch Dermatol Res 289, 623-630, doi:10.1007/s004030050251 (1997).
41. Arts, R. J. et al. Glutaminolysis and Fumarate Accumulation Integrate Immunometabolic and Epigenetic Programs in Trained Immunity. Cell Metab 24, 807-819, doi:10.1016/j.cmet.2016.10.008 (2016).
42. Ryan, D. G. et al. Nrf2 activation reprograms macrophage intermediary metabolism and suppresses the type I interferon response. iScience 25, 103827, doi:https://doi.org/10.1016/j.isci.2022.103827 (2022).
43. Shanmugasundaram, K. et al. The oncometabolite fumarate promotes pseudohypoxia through noncanonical activation of NF-kappaB signaling. J Biol Chem 289, 24691-24699, doi:10.1074/jbc.M114.568162 (2014).
44. West, A. P. et al. Mitochondrial DNA stress primes the antiviral innate immune response. Nature 520, 553-557, doi:10.1038/nature14156 (2015).
45. Sliter, D. A. et al. Parkin and PINK1 mitigate STING-induced inflammation. Nature 561, 258-262, doi:10.1038/s41586-018-0448-9 (2018).
46. McArthur, K. et al. BAK/BAX macropores facilitate mitochondrial herniation and mtDNA efflux during apoptosis. Science 359, doi:10.1126/science.aao6047 (2018).
47. Dang, E. V., McDonald, J. G., Russell, D. W. & Cyster, J. G. Oxysterol Restraint of Cholesterol Synthesis Prevents AIM2 Inflammasome Activation. Cell 171, 1057-1071 e1011, doi:10.1016/j.cell.2017.09.029 (2017).
48. Haag, S. M. et al. Targeting STING with covalent small-molecule inhibitors. Nature 559, 269-273, doi:10.1038/s41586-018-0287-8 (2018).
49. Stunz, L. L. et al. Inhibitory oligonucleotides specifically block effects of stimulatory CpG oligonucleotides in B cells. Eur J Immunol 32, 1212-1222, doi:10.1002/1521-4141(200205)32: 5<1212::AID-IMMU1212>3.0.CO;2-D (2002).
50. Prantner, D. et al. 5,6-Dimethylxanthenone-4-acetic acid (DMXAA) activates stimulator of interferon gene (STING)-dependent innate immune pathways and is regulated by mitochondrial membrane potential. J Biol Chem 287, 39776-39788, doi:10.1074/jbc.M112.382986 (2012).
51. Dhir, A. et al. Mitochondrial double-stranded RNA triggers antiviral signalling in humans. Nature 560, 238-242, doi:10.1038/s41586-018-0363-0 (2018).
52. Tigano, M., Vargas, D. C., Tremblay-Belzile, S., Fu, Y. & Sfeir, A. Nuclear sensing of breaks in mitochondrial DNA enhances immune surveillance. Nature 591, 477-481, doi:10.1038/s41586-021-03269-w (2021).
53 Kariko, K., Buckstein, M., Ni, H. & Weissman, D. Suppression of RNA recognition by Toll-like receptors: the impact of nucleoside modification and the evolutionary origin of RNA. Immunity 23, 165-175, doi:10.1016/j.immuni.2005.06.008 (2005).
54. Rai, P. et al. IRGM1 links mitochondrial quality control to autoimmunity. Nat Immunol 22, 312-321, doi:10.1038/s41590-020-00859-0 (2021).
55. Kruger, A. et al. Human TLR8 senses UR/URR motifs in bacterial and mitochondrial RNA. EMBO Rep 16, 1656-1663, doi:10.15252/embr.201540861 (2015).
56. Pichlmair, A. et al. RIG-I-mediated antiviral responses to single-stranded RNA bearing 5′-phosphates. Science 314, 997-1001, doi:10.1126/science. 1132998 (2006).
57. Koshiba, T., Yasukawa, K., Yanagi, Y. & Kawabata, S. Mitochondrial membrane potential is required for MAVS-mediated antiviral signaling. Sci Signal 4, ra7, doi:10.1126/scisignal.2001147 (2011).
58. Kim, S. et al. Mitochondrial double-stranded RNAs govern the stress response in chondrocytes to promote osteoarthritis development. Cell Rep 40, 111178, doi:10.1016/j.celrep.2022.111178 (2022).
59. Rasa, S. M. M. A., F.; Krepelova, A.; Nunna, S.; Omrani, O.; Gebert, N.; Adam, L.; Käppel, S.; Höhn, S.; Donati, G.; Jurkowski, T.P.; Rudolph, K.L.; Ori, A.; Neri, F. Inflammaging is driven by upregulation of innate immune receptors and systemic interferon signaling and is ameliorated by dietary restriction. Cell Rep 39 (2022).
60. Buskiewicz, I. A. et al. Reactive oxygen species induce virus-independent MAVS oligomerization in systemic lupus erythematosus. Sci Signal 9, ra115, doi:10.1126/scisignal.aaf1933 (2016).
61. Ruiz-Limon, P. et al. Atherosclerosis and cardiovascular disease in systemic lupus erythematosus: effects of in vivo statin treatment. Ann Rheum Dis 74, 1450-1458, doi:10.1136/annrheumdis-2013-204351 (2015).
62. Davis, P., Cunnington, P. & Hughes, G. R. Double-stranded RNA antibodies in systemic lupus erythematosus. Ann Rheum Dis 34, 239-243, doi:10.1136/ard.34.3.239 (1975).
63. Caielli, S. et al. Oxidized mitochondrial nucleoids released by neutrophils drive type I interferon production in human lupus. J Exp Med 213, 697-713, doi:10.1084/jem.20151876 (2016).
64. Sarkar, P. et al. Reduced expression of mitochondrial fumarate hydratase in progressive multiple sclerosis contributes to impaired in vitro mesenchymal stromal cell-mediated neuroprotection. Mult Scler 28, 1179-1188, doi:10.1177/13524585211060686 (2022).
65. Li, Q. et al. RNA editing underlies genetic risk of common inflammatory diseases. Nature, doi:10.1038/s41586-022-05052-x (2022).
66. Pang, Z. et al. MetaboAnalyst 5.0: narrowing the gap between raw spectra and functional insights. Nucleic Acids Res 49, W388-W396, doi:10.1093/nar/gkab382 (2021).
67. Anders, S. & Huber, W. Differential expression analysis for sequence count data. Genome Biol 11, R106, doi:10.1186/gb-2010-11-10-r106 (2010).
68. Shah, A. D., Goode, R. J. A., Huang, C., Powell, D. R. & Schittenhelm, R. B. LFQ-Analyst: An Easy-To-Use Interactive Web Platform To Analyze and Visualize Label-Free Proteomics Data Preprocessed with MaxQuant. J Proteome Res 19, 204-211, doi:10.1021/acs.jproteome.9b00496 (2020).
69. Kuleshov, M. V. et al. Enrichr: a comprehensive gene set enrichment analysis web server 2016 update. Nucleic Acids Res 44, W90-97, doi:10.1093/nar/gkw377 (2016).
70. Subramanian, A. et al. Gene set enrichment analysis: a knowledge-based approach for interpreting genome-wide expression profiles. Proc Natl Acad Sci USA 102, 15545-15550, doi:10.1073/pnas.0506580102 (2005).

We claim:

1. A method for prevention and/or treatment of inflammation in a patient in need thereof, the method comprising
administering an effective amount of a fumarate hydratase modulating agent to said patient.

2. The method of claim 1 wherein the inflammation is interferon driven inflammation.

3. The method of claim 1 wherein said patient has low levels of fumarate hydratase expression compared to a healthy subject.

4. The method of claim 1 wherein said patient has an interferon-driven disease associated with decreased FH activity and/or expression.

5. The method of claim 1 wherein the fumarate hydratase modulating agent upregulates fumarate hydratase activity and/or expression to reduce or prevent macrophage, cytokine and/or interferon production in a patient in need thereof.

6. The method of claim 1 wherein the fumarate hydratase modulating agent upregulates fumarate hydratase activity and/or expression to reduce or prevent type 1 interferon (type 1 IFN) production in a patient in need thereof.

7. The method of claim 6 in which the type 1 interferon (type 1 IFN) is IFN-β.

8. The method of claim 1 wherein the fumarate hydratase modulating agent upregulates fumarate hydratase activity and/or expression to reduce or prevent interleukin-10 (IL-10) and/or tumor necrosis factor (TNF) production in a patient in need thereof.

9. The method of claim 1 wherein the fumarate hydratase modulating agent upregulates fumarate hydratase activity and/or expression to reduce or prevent cytosolic mitochondrial RNA release and/or signalling via ssRNA and dsRNA receptors, including ssRNA receptor TLR7 and/or dsRNA receptors MDA5 and RIG-I.

10. The method of claim 1 wherein the fumarate hydratase modulating agent is selected from one or more of the following:
fumarate hydratase;
a small molecule activator of fumarate hydratase;
a pharmaceutical composition comprising fumarate hydratase or a small molecule activator of fumarate hydratase;
a viral vector comprising a fumarate hydratase gene or mRNA thereof; or
a non-viral vector, such as a lipid nanoparticle, comprising a fumarate hydratase gene or mRNA thereof.

11. The method of claim 1 wherein the method comprises the delivery of the fumarate hydratase modulating agent to inflammatory cells or pro-inflammatory cells of said patient.

12. The method of claim 1 wherein the method comprises the delivery of the fumarate hydratase gene or mRNA thereof to inflammatory cells or pro-inflammatory cells of said patient.

13. The method of claim 11 wherein the inflammatory or pro-inflammatory cells include macrophages.

14. The method of claim 1 for the treatment of inflammation of the skin, kidneys and/or joints in said patient.

15. The method of claim 1 for the treatment of auto-immune diseases such as systemic lupus erythematosus (SLE), asthma, arthritis, sepsis and covid-19 in said patient.

16. The method of claim 1 for the treatment of chronic inflammation in said patient.

17. The method of claim 1 for the treatment of sepsis during bacterial or viral infections in said patient.

18. An anti-inflammatory agent for use in upregulating fumarate hydratase activity and/or expression to reduce or prevent interferon production, wherein the anti-inflammatory agent is selected from one or more of the following:
fumarate hydratase;
a small molecule activator of fumarate hydratase;
a pharmaceutical composition comprising said small molecule activator of fumarate hydratase;
a viral vector comprising a fumarate hydratase gene or mRNA thereof; or
a non-viral vector, such as a lipid nanoparticle, comprising a fumarate hydratase gene or mRNA thereof.


SEQUENCE LISTING (1 sequence)

SEQ ID NO: 1 - protein, 510 aa, Homo sapiens
MYRALRLLARSRPLVRAPAAALASAPGLGGAAVPSFWPPNAARMASQNSFRIEYDTFGELKVPNDKYYGAQTVRSTMNFKIGGVTERMPTPVIKAFGILKRAAAEVNQDYGLDPKIANAIMKAADEVAEGKLNDHFPLVVWQTGSGTQTNMNVNEVISNRAIEMLGGELGSKIPVHPNDHVNKSQSSNDTFPTAMHIAAAIEVHEVLLPGLQKLHDALDAKSKEFAQIIKIGRTHTQDAVPLTLGQEFSGYVQQVKYAMTRIKAAMPRIYELAAGGTAVGTGLNTRIGFAEKVAAKVAALTGLPFVTAPNKFEALAAHDALVELSGAMNTTACSLMKIANDIRFLGSGPRSGLGELILPENEPGSSIMPGKVNPTQCEAMTMVAAQVMGNHVAVTVGGSNGHFELNVFKPMMIKNVLHSARLLGDASVSFTENCVVGIQANTERINKLMNESLMLVTALNPHIGYDKAAKIAKTAHKNGSTLKETAIELGYLTAEQFDEWVKPKDMLGPK